SEQUENCE LISTING (89 sequences)

SEQ ID NO: 1 - DNA, 2856 bp, Homo sapiens
  CDS (591)..(2216): ASCT2 nucleotide sequence GenBank Accession number NM_005628
gtaaccgctactcccggacaccagaccaccgccttccgtacacaggggcccgcatcccaccctcccggacctaagagcctgggtcccctgtttccggaggtccgcttcccggcccccagattctggcatcccagccctcagtgtccaagacccaggcagcccgggtccccgcctcccggatccaggcgtccgggatctgcgccaccagaacctagcctcctgcagacctccgccatctgggggcactcaacctcctggagccaagggccccacgtcccacccagagaaactctcgtattcccagctcctagggccaaggaacccgggcgctccgaactcccagctttcggacatctggcacacggggcagagcagagaagctcagcgcccagcctggggaatttaaacactccagcttccaagagccaaggaacttcagtgctgtgaactcacaactctaaggagccctccaaagttccagtctccaggtgctgttactcaactcagtcctaggaacgtcgggtcctgggaaggagcccaagcgctcccagccagcttccaggcgctaagaaaccccggtgcttcccatcatggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtacgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattatgtggaccgtacggagtcgagaagcacagagcctgagttgatacaagtgaagagtgagctgcccctggatccgctgccagtccccactgaggaaggaaaccccctcctcaaacactatcgggggcccgcaggggatgccacggtcgcctctgagaaggaatcagtcatgtaaaccccgggagggaccttccctgccctgctgggggtgctctttggacactggattatgaggaatggataaatggatgagctagggctctgggggtctgcctgcacactctggggagccaggggccccagcaccctccaggacaggagatctgggatgcctggctgctggagtacatgtgttcacaagggttactcctcaaaacccccagttctcactcatgtccccaactcaaggctagaaaacagcaagatggagaaataatgttctgctgcgtccccaccgtgacctgcctggcctcccctgtctcagggagcaggtcacaggtcaccatggggaattctagcccccactggggggatgttacaacaccatgctggttattttggcggctgtagttgtggggggatgtgtgtgtgcacgtgtgtgtgtgtgtgtgtgtgtgtgtgtgtgtgtgttctgtgacctcctgtccccatggtacgtcccaccctgtccccagatcccctattccctccacaataacagaaacactcccagggactctggggagaggctgaggacaaatacctgctgtcactccagaggacattttttttagcaataaaattgagtgtcaactatttaaaaaaaaaaaaaaaaa

SEQ ID NO: 2 - protein, 541 aa, Homo sapiens
  PEPTIDE (1)..(541): ASCT2 peptide sequence GenPept Accession number NP_005619
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVM

SEQ ID NO: 3 - DNA, 21 bp, Artificial
  Description of Artificial sequence ASCT2 primer (Fw#1)
gataaatggatgagctagggc

SEQ ID NO: 4 - DNA, 21 bp, Artificial
  Description of Artificial sequence ASCT2 primer (Rv#1)
gttattgtggagggaataggg

SEQ ID NO: 5 - DNA, 1689 bp, Artificial
  Description of Artificial sequence ASCT2-myc/His sequence
atggtggccgatcctcctcgagactccaaggggctcgcagcggcggagcccaccgccaacgggggcctggcgctggcctccatcgaggaccaaggcgcggcagcaggcggctactgcggttcccgggaccaggtgcgccgctgccttcgagccaacctgcttgtgctgctgacagtggtggccgtggtggccggcgtggcgctgggactgggggtgtcgggggccgggggtgcgctggcgttgggcccggagcgcttgagcgccttcgtcttcccgggcgagctgctgctgcgtctgctgcggatgatcatcttgccgctggtggtgtgcagcttgatcggcggcgccgccagcctggaccccggcgcgctcggccgtctgggcgcctgggcgctgctctttttcctggtcaccacgctgctggcgtcggcgctcggagtgggcttggcgctggctctgcagccgggcgccgcctccgccgccatcaacgcctccgtgggagccgcgggcagtgccgaaaatgcccccagcaaggaggtgctcgattcgttcctggatcttgcgagaaatatcttcccttccaacctggtgtcagcagcctttcgctcatactctaccacctatgaagagaggaatatcaccggaaccagggtgaaggtgcccgtggggcaggaggtggaggggatgaacatcctgggcttggtagtgtttgccatcgtctttggtgtggcgctgcggaagctggggcctgaaggggagctgcttatccgcttcttcaactccttcaatgaggccaccatggttctggtctcctggatcatgtggtacgcccctgtgggcatcatgttcctggtggctggcaagatcgtggagatggaggatgtgggtttactctttgcccgccttggcaagtacattctgtgctgcctgctgggtcacgccatccatgggctcctggtactgcccctcatctacttcctcttcacccgcaaaaacccctaccgcttcctgtggggcatcgtgacgccgctggccactgcctttgggacctcttccagttccgccacgctgccgctgatgatgaagtgcgtggaggagaataatggcgtggccaagcacatcagccgtttcatcctgcccatcggcgccaccgtcaacatggacggtgccgcgctcttccagtgcgtggccgcagtgttcattgcacagctcagccagcagtccttggacttcgtaaagatcatcaccatcctggtcacggccacagcgtccagcgtgggggcagcgggcatccctgctggaggtgtcctcactctggccatcatcctcgaagcagtcaacctcccggtcgaccatatctccttgatcctggctgtggactggctagtcgaccggtcctgtaccgtcctcaatgtagaaggtgacgctctgggggcaggactcctccaaaattacgtggaccgtacggagtcgagaagcacagagcctgagttgatacaagtgaagagtgagctgcccctggatccgctgccagtccccactgaggaaggaaaccccctcctcaaacactatcgggggcccgcaggggatgccacggtcgcctctgagaaggaatcagtcatggaacaaaaactcatctcagaagaggatctgaatatgcataccggtcatcatcaccatcaccattga

SEQ ID NO: 6 - protein, 562 aa, Artificial
  Description of Artificial sequence ASCT2-myc/His sequence
MVADPPRDSKGLAAAEPTANGGLALASIEDQGAAAGGYCGSRDQVRRCLRANLLVLLTVVAVVAGVALGLGVSGAGGALALGPERLSAFVFPGELLLRLLRMIILPLVVCSLIGGAASLDPGALGRLGAWALLFFLVTTLLASALGVGLALALQPGAASAAINASVGAAGSAENAPSKEVLDSFLDLARNIFPSNLVSAAFRSYSTTYEERNITGTRVKVPVGQEVEGMNILGLVVFAIVFGVALRKLGPEGELLIRFFNSFNEATMVLVSWIMWYAPVGIMFLVAGKIVEMEDVGLLFARLGKYILCCLLGHAIHGLLVLPLIYFLFTRKNPYRFLWGIVTPLATAFGTSSSSATLPLMMKCVEENNGVAKHISRFILPIGATVNMDGAALFQCVAAVFIAQLSQQSLDFVKIITILVTATASSVGAAGIPAGGVLTLAIILEAVNLPVDHISLILAVDWLVDRSCTVLNVEGDALGAGLLQNYVDRTESRSTEPELIQVKSELPLDPLPVPTEEGNPLLKHYRGPAGDATVASEKESVMEQKLISEEDLNMHTGHHHHHH

SEQ ID NO: 7 - DNA, 100 bp, Artificial
  Description of Artificial sequence N-ASCT2 primer (#1) sequence
aaggaaaaaagaattcaaaccccggtgcttcccatcatggtggccgatcctcctcgagactccaaggggctcgcagcggcggagcccaccgccaacgggg

SEQ ID NO: 8 - DNA, 140 bp, Artificial
  Description of Artificial sequence N-ASCT2 primer (#2) sequence
cggccaccactgtcagcagcacaagcaggttggctcgaaggcagcggcgcacctggtcccgggaaccgcagtagccgcctgctgccgcgccttggtcctcgatggaggccagcgccaggcccccgttggcggtgggctcc

SEQ ID NO: 9 - DNA, 130 bp, Artificial
  Description of Artificial sequence N-ASCT2 primer (#3) sequence
gctgctgacagtggtggccgtggtggccggcgtggcgctgggactgggggtgtcgggggccgggggtgcgctggcgttgggcccggagcgcttgagcgccttcgtcttcccgggcgagctgctgctgcgt

SEQ ID NO: 10 - DNA, 100 bp, Artificial
  Description of Artificial sequence N-ASCT2 primer (#4) sequence
ccgagcgcgccggggtccaggctggcggcgccgccgatcaagctgcacaccaccagcggcaagatgatcatccgcagcagacgcagcagcagctcgcccg

SEQ ID NO: 11 - DNA, 20 bp, Artificial
  Description of Artificial sequence C-ASCT2-myc/His primer (Fw#2) sequence
ctggaccccggcgcgctcgg

SEQ ID NO: 12 - DNA, 120 bp, Artificial
  Description of Artificial sequence C-ASCT2-myc/His primer (Rv#2) sequence
cgatggtacctcaatggtgatggtgatgatgaccggtatgcatattcagatcctcttctgagatgagtttttgttccatgactgattccttctcagaggcgaccgtggcatcccctgcgg

SEQ ID NO: 13 - DNA, 40 bp, Artificial
  Description of Artificial sequence C-ASCT2-myc/His primer (Rv#3) sequence
ggactagtgcggccgcgatggtacctcaatggtgatggtg

SEQ ID NO: 14 - protein, 16 aa, Artificial
  Description of Artificial sequence ASCT2 peptide (2-16, Cys) sequence
VADPPRDSKGLAAAEC

SEQ ID NO: 15 - DNA, 25 bp, Artificial
  Description of Artificial sequence primer (mG3a2) sequence
ctggacagggctccatagttccatt

SEQ ID NO: 16 - DNA, 30 bp, Artificial
  Description of Artificial sequence primer (mG2ba1) sequence
ttgaccaggcatcccagagtcacggaggaa

SEQ ID NO: 17 - DNA, 32 bp, Artificial
  Description of Artificial sequence primer (mKa1) sequence
ctaacactcattcctgttgaagctcttgacaa

SEQ ID NO: 18 - DNA, 417 bp, Mus musculus
  CDS (1)..(417): KM4008VH nucleotide sequence
atgggttggctgtggaacttgctattcctgatggcagctgcccaaagtgcccaagcacagatccagttggtacagtctggacctgagctgaagaagcctggagagacagtcaagatctcctgcaaggcttctgggtataccttcacaacctttggaatgagctgggtgaaacaggttccaggaaagggtttaaagtggatgggctggatacacacctacgctggagtgccaatatatggtgatgacttcaagggacggtttgccttctctttggaaacctctgccagcactgcctatttgcagatcaacaacgtcaaaaatgaggacacggctacatatttctgtgcacgacgttcggataattaccgttatttttttgactattggggccaaggcaccactctcacagtctcctcg

SEQ ID NO: 19 - protein, 139 aa, Mus musculus
  PEPTIDE (1)..(139): KM4008VH peptide sequence
MGWLWNLLFLMAAAQSAQAQIQLVQSGPELKKPGETVKISCKASGYTFTTFGMSWVKQVPGKGLKWMGWIHTYAGVPIYGDDFKGRFAFSLETSASTAYLQINNVKNEDTATYFCARRSDNYRYFFDYWGQGTTLTVSS

SEQ ID NO: 20 - DNA, 384 bp, Mus musculus
  CDS (1)..(384): KM4008VL nucleotide sequence
atgatgtcctctgctcggttccttggtctcctgttgctctgctttcaaggtaccagatgtgatatccagatgacacagactacatcctccctgtctgcctctctgggagacagagtcaccatcagttgcagggcaagtcaggacattaggaattatttaaactggtatcaacgtaaaccacatggaactgttaaactcctgatctactacacatcaagattacactcaggagtcccgtcaaggttcagtggcagtgggtctggaacagattattctctcaccattagcaacctggaacaagaagatattgccacttacttttgccaacagggtcatacgcttcctccgacgttcggtggaggcaccaagttggaaatcaaacgg

SEQ ID NO: 21 - protein, 128 aa, Mus musculus
  PEPTIDE (1)..(128): KM4008VL peptide sequence
MMSSARFLGLLLLCFQGTRCDIQMTQTTSSLSASLGDRVTISCRASQDIRNYLNWYQRKPHGTVKLLIYYTSRLHSGVPSRFSGSGSGTDYSLTISNLEQEDIATYFCQQGHTLPPTFGGGTKLEIKR

SEQ ID NO: 22 - DNA, 417 bp, Mus musculus
  CDS (1)..(417): KM4012VH nucleotide sequence
atgggttggctgtggaacttgctattcctgatggcagctgcccaaagtgcccaagcacagatccagttggtacagtctggacctgagctgaagaagcctggagagacagtcaagatctcctgcaaggcttctggatataccttcacgacctatggaatgagctgggtgaaacagactccaggaaagggtttaaagtggatgggctggatacacacctactctggagtgccaatatatggtgatgactccaagggacggtttgccttctctttggaaacctctggcagcattgcctatttgcagatcaacaacgtcaaaaatgaggacacggctacatatttctgtgcaagacgttcggatggttaccgttacttctttgactattggggccaaggcaccactctcacagtctcctca

SEQ ID NO: 23 - protein, 139 aa, Mus musculus
  PEPTIDE (1)..(139): KM4012VH peptide sequence
MGWLWNLLFLMAAAQSAQAQIQLVQSGPELKKPGETVKISCKASGYTFTTYGMSWVKQTPGKGLKWMGWIHTYSGVPIYGDDSKGRFAFSLETSGSIAYLQINNVKNEDTATYFCARRSDGYRYFFDYWGQGTTLTVSS

SEQ ID NO: 24 - DNA, 384 bp, Mus musculus
  CDS (1)..(384): KM4012VL nucleotide sequence
atgatgtcctctgctcagttccttggtctcctgttgctctgctttcaaggtaccagatgtgatatccagatgacacagattacatcctccctgtctgcctctctgggagacagagtcaccatcagttgcagggcaagtcaggacattaggaattatttaaactggtatcagcagaaaccagatggaactgttaaactcctgatctactacacatcaagattacactcaggagtcccatcaaggttcagtggcagtgggtctggaacagattattctctcaccattagcaacctggaacaagaagatattgccacttacttttgccaacagggtaatacgcttcctccgacgttcggtggaggcaccaagttggaaatcaaacgg

SEQ ID NO: 25 - protein, 128 aa, Mus musculus
  PEPTIDE (1)..(128): KM4012VL peptide sequence
MMSSAQFLGLLLLCFQGTRCDIQMTQITSSLSASLGDRVTISCRASQDIRNYLNWYQQKPDGTVKLLIYYTSRLHSGVPSRFSGSGSGTDYSLTISNLEQEDIATYFCQQGNTLPPTFGGGTKLEIKR

SEQ ID NO: 26 - protein, 5 aa, Mus musculus
  PEPTIDE (1)..(5): KM4008H CDR1 peptide sequence
TFGMS

SEQ ID NO: 27 - protein, 17 aa, Mus musculus
  PEPTIDE (1)..(17): KM4008 CDR2 peptide sequence
WIHTYAGVPIYGDDFKG

SEQ ID NO: 28 - protein, 11 aa, Mus musculus
  PEPTIDE (1)..(11): KM4008H CDR3 peptide sequence
RSDNYRYFFDY

SEQ ID NO: 29 - protein, 11 aa, Mus musculus
  PEPTIDE (1)..(11): KM4008L CDR1 peptide sequence
RASQDIRNYLN

SEQ ID NO: 30 - protein, 7 aa, Mus musculus
  PEPTIDE (1)..(7): KM4008L CDR2 peptide sequence
YTSRLHS

SEQ ID NO: 31 - protein, 9 aa, Mus musculus
  PEPTIDE (1)..(9): KM4008L CDR3 peptide sequence
QQGHTLPPT

SEQ ID NO: 32 - protein, 5 aa, Mus musculus
  PEPTIDE (1)..(5): KM4012H CDR1 peptide sequence
TYGMS

SEQ ID NO: 33 - protein, 17 aa, Mus musculus
  PEPTIDE (1)..(17): KM4012H CDR2 peptide sequence
WIHTYSGVPIYGDDSKG

SEQ ID NO: 34 - protein, 11 aa, Mus musculus
  PEPTIDE (1)..(11): KM4012H CDR3 peptide sequence
RSDGYRYFFDY

SEQ ID NO: 35 - protein, 11 aa, Mus musculus
  PEPTIDE (1)..(11): KM4012L CDR1 peptide sequence
RASQDIRNYLN

SEQ ID NO: 36 - protein, 7 aa, Mus musculus
  PEPTIDE (1)..(7): KM4012L CDR2 peptide sequence
YTSRLHS

SEQ ID NO: 37 - protein, 9 aa, Mus musculus
  PEPTIDE (1)..(9): KM4012L CDR3 peptide sequence
QQGNTLPPT

SEQ ID NO: 38 - DNA, 40 bp, Artificial
  Description of Artificial sequence cKM4008VH/c4012VH primer (Fw) sequence
aaaagcggccgcccactgagcccaagtcttagacatcatg

SEQ ID NO: 39 - DNA, 40 bp, Artificial
  Description of Artificial sequence cKM4008VH primer (Rv) sequence
cgatgggcccttggtggaagccgaggagactgtgagagtg

SEQ ID NO: 40 - DNA, 40 bp, Artificial
  Description of Artificial sequence cKM4008VL/cKM4012VL primer (Fw) sequence
ccggaattcattgaagtcaagactcagcctggacatgatg

SEQ ID NO: 41 - DNA, 40 bp, Artificial
  Description of Artificial sequence cKM4008VL/cKM4012VL primer (Rv) sequence
ccaccgtacgtttgatttccaacttggtgcctccaccgaa

SEQ ID NO: 42 - DNA, 40 bp, Artificial
  Description of Artificial sequence cKM4012VH primer (Rv) sequence
cgatgggcccttggtggaggctgaggagactgtgagagtg

SEQ ID NO: 43 - DNA, 26 bp, Artificial
  Description of Artificial sequence primer (rG2a) sequence
ccacaaggattgcattcccttggcac

SEQ ID NO: 44 - DNA, 29 bp, Artificial
  Description of Artificial sequence primer (rKa2) sequence
cctgttgaagctcttgacgacgggtgagg

SEQ ID NO: 45 - DNA, 426 bp, Rattus norvegicus
  CDS (1)..(426): KM4018VH nucleotide sequence
atggacatcaggctcagcttggctttccttgtccttttcataaaaggtgtccagtgtgaggtgcagctggtggagtctgggggaggcttagtgcagtctggaaggtccataagactctcctgtgcagcctcaggattctctttcagtaactattacatggcctgggtccgccaggctccatcgaagggtctggagtgggtcgcatccattactaaaggtggtggtaatacttactatcgagactccgtgaagggccgattcactttctccagagataatgcaaaaagcaccctatatttgcaaatggacagtctgaggtctgaggacacggccacttattactgtgcaagacaggttactatagcagctgtatctacctcctactttgattcctggggccaaggagtcatggtcacagtctcctca

SEQ ID NO: 46 - protein, 142 aa, Rattus norvegicus
  PEPTIDE (1)..(142): KM4018VH peptide sequence
MDIRLSLAFLVLFIKGVQCEVQLVESGGGLVQSGRSIRLSCAASGFSFSNYYMAWVRQAPSKGLEWVASITKGGGNTYYRDSVKGRFTFSRDNAKSTLYLQMDSLRSEDTATYYCARQVTIAAVSTSYFDSWGQGVMVTVSS

SEQ ID NO: 47 - DNA, 393 bp, Rattus norvegicus
  CDS (1)..(393): KM4018VL nucleotide sequence
atgaagacagacacactcctgctgtgggctctgctgctctgggttccaggttgcactggtgacattgtgctgacccagtctcctgctttggctgtgtctctagggcagagggccaccatctcctgcaagaccaatcagaaggtcgattattatggcaatagttatgtgtactggtaccaacagaaaccaggacaacaacccaaactcctcatctatttagcatccaacttagcatctgggatccctgccaggttcagtggtagagggtctgggacagacttcaccctcaccattgatcctgtggaggctgatgatactgcaacctattactgtcagcagagtaggaatcttccgtacacgtttggagctgggaccaagctggaactgaaacgg

SEQ ID NO: 48 - protein, 131 aa, Rattus norvegicus
  PEPTIDE (1)..(131): KM4018VL peptide sequence
MKTDTLLLWALLLWVPGCTGDIVLTQSPALAVSLGQRATISCKTNQKVDYYGNSYVYWYQQKPGQQPKLLIYLASNLASGIPARFSGRGSGTDFTLTIDPVEADDTATYYCQQSRNLPYTFGAGTKLELKR

SEQ ID NO: 49 - protein, 5 aa, Rattus norvegicus
  PEPTIDE (1)..(5): KM4018H CDR1 peptide sequence
NYYMA

SEQ ID NO: 50 - protein, 17 aa, Rattus norvegicus
  PEPTIDE (1)..(17): KM4018H CDR2 peptide sequence
SITKGGGNTYYRDSVKG

SEQ ID NO: 51 - protein, 14 aa, Rattus norvegicus
  PEPTIDE (1)..(14): KM4018H CDR3 peptide sequence
QVTIAAVSTSYFDS

SEQ ID NO: 52 - protein, 15 aa, Rattus norvegicus
  PEPTIDE (1)..(15): KM4018L CDR1 peptide sequence
KTNQKVDYYGNSYVY

SEQ ID NO: 53 - protein, 7 aa, Rattus norvegicus
  PEPTIDE (1)..(7): KM4018L CDR2 peptide sequence
LASNLAS

SEQ ID NO: 54 - protein, 9 aa, Rattus norvegicus
  PEPTIDE (1)..(9): KM4018L CDR3 peptide sequence
QQSRNLPYT

SEQ ID NO: 55 - DNA, 40 bp, Artificial
  Description of Artificial sequence cKM4018 VH primer (Fw) sequence
aaaagcggccgctgcagcactgcacagactacgcaccatg

SEQ ID NO: 56 - DNA, 40 bp, Artificial
  Description of Artificial sequence cKM4018 VH primer (Rv) sequence
cgatgggcccttggtggaagctgaggagactgtgaccatg

SEQ ID NO: 57 - DNA, 40 bp, Artificial
  Description of Artificial sequence cKM4018 VL primer (Fw) sequence
ccggaattcccagcctctctttcagctgtcagagatgaag

SEQ ID NO: 58 - DNA, 40 bp, Artificial
  Description of Artificial sequence cKM4018 VL primer (Rv) sequence
ccaccgtacgtttcagttccagcttggtcccagctccaaa

SEQ ID NO: 59 - DNA, 1731 bp, Artificial
  CDS (1)..(1731): Description of Artificial sequence mouse ASCT2-myc/His sequence
atggcagtggatccccctaaggctgaccccaaaggggtagtagcggtggattccaccgcgaacggtggccccgcgctgggatccagagaggaccagagtgcgaaagcaggtggttgctgcggttcccgtgaccgggtgcgccgctgcattcgcgccaacctgctggtgctgctcacggtggctgcggtggtggctggcgtggggctggggctgggggtctcggcggcgggcggtgctgacgcgctgggtcccgcgcgcttgaccgctttcgccttcccgggagagctgctgctgcgtctgctgaagatgatcatcctgccgctcgtggtgtgcagcctgatcggaggtgcagccagcttggaccctagcgcgctcggtcgtgtgggcgcctgggcgctgctctttttcctggtcaccacactgctcgcgtcggcgctcggcgtgggtttggccctggcgctgaagccgggcgccgccgttaccgccatcacctccatcaacgactctgttgtagacccctgtgcccgcagtgcaccaaccaaagaggtgctggattcctttctagatctcgtgaggaatattttcccctccaatctggtgtctgctgccttccgctcttttgctacctcatatgaacccaaagacaactcatgtaaaataccgcaatcctgtatccagcgggagatcaattcaacgatggtccagcttctctgtgaggtggagggaatgaacatcctgggcctggtggtcttcgctatcgtctttggtgtggctctgcggaagctggggcccgagggtgagctgctcattcgtttcttcaactccttcaatgatgccaccatggtcctggtctcctggattatgtggtacgcacccgttggaatcctgttcctggtggccagcaagattgtggagatgaaagacgtgcgccagctcttcatcagcctcggcaaatacattctgtgctgcctgctgggccacgccatccacgggctcctggttctgcctctcatctacttcctcttcacccgcaaaaatccctatcgattcctgtggggcatcatgacacccctggccactgctttcgggacctcttctagctctgccaccttgcctctgatgatgaagtgtgtagaggagaagaatggtgtggccaaacacatcagccggttcatcctacccatcggcgccacggtcaacatggacggggcggcgctgttccagtgtgtggcggcagtgttcatcgcacaactaaacggggtgtccctggacttcgtgaagatcatcaccatcctggtcacagccacagcatccagtgtcggtgcggcagggatccctgcagggggcgtcctcaccctcgccatcatcctggaagcagtcagcctgcctgtgaaggacatctccttgatcttggccgtggactggctagtggacaggtcctgtactgtcctcaacgtggaaggtgatgcttttggggctggactgcttcagagttacgtggatcgaaccaagatgccgagctcagagcccgaattgatccaggtgaagaacgaggtgtctctgaatccactgcccctcgccacagaggaggggaatcccctcctgaaacagtaccagggacccaccggggactccagtgccacgttcgaaaaggaatctgtcatggaacaaaaactcatctcagaagaggatctgaatatgcataccggtcatcatcaccatcaccattga

SEQ ID NO: 60 - protein, 576 aa, Artificial
  PEPTIDE (1)..(576): Description of Artificial sequence mouse ASCT2-myc/His sequence
MAVDPPKADPKGVVAVDSTANGGPALGSREDQSAKAGGCCGSRDRVRRCIRANLLVLLTVAAVVAGVGLGLGVSAAGGADALGPARLTAFAFPGELLLRLLKMIILPLVVCSLIGGAASLDPSALGRVGAWALLFFLVTTLLASALGVGLALALKPGAAVTAITSINDSVVDPCARSAPTKEVLDSFLDLVRNIFPSNLVSAAFRSFATSYEPKDNSCKIPQSCIQREINSTMVQLLCEVEGMNILGLVVFAIVFGVALRKLGPEGELLIRFFNSFNDATMVLVSWIMWYAPVGILFLVASKIVEMKDVRQLFISLGKYILCCLLGHAIHGLLVLPLIYFLFTRKNPYRFLWGIMTPLATAFGTSSSSATLPLMMKCVEEKNGVAKHISRFILPIGATVNMDGAALFQCVAAVFIAQLNGVSLDFVKIITILVTATASSVGAAGIPAGGVLTLAIILEAVSLPVKDISLILAVDWLVDRSCTVLNVEGDAFGAGLLQSYVDRTKMPSSEPELIQVKNEVSLNPLPLATEEGNPLLKQYQGPTGDSSATFEKESVMEQKLISEEDLNMHTGHHHHHH

SEQ ID NO: 61 - DNA, 59 bp, Artificial
  Description of Artificial sequence mouse ASCT2-myc/His primer (Fw) sequence
ccggaattcacggctcagcatggcagtggatccccctaaggctgaccccaaaggggtag

SEQ ID NO: 62 - DNA, 120 bp, Artificial
  Description of Artificial sequence mouse ASCT2-myc/His primer (Rv) sequence
cgatggtacctcaatggtgatggtgatgatgaccggtatgcatattcagatcctcttctgagatgagtttttgttccatgacagattccttttcgaacgtggcactggagtccccggtgg

SEQ ID NO: 63 - DNA, 28 bp, Artificial
  Description of Artificial sequence human ASCT2 NcoI primer (Fw) sequence
tgggtcacgccattcatgggctcctggt

SEQ ID NO: 64 - DNA, 28 bp, Artificial
  Description of Artificial sequence human ASCT2 NcoI primer (Rv) sequence
accaggagcccatgaatggcgtgaccca

SEQ ID NO: 65 - DNA, 36 bp, Artificial
  Description of Artificial sequence human ASCT2 BamIII primer (Fw) sequence
cccgcaaaaacccctatcgattcctgtggggcatcg

SEQ ID NO: 66 - DNA, 36 bp, Artificial
  Description of Artificial sequence human ASCT2 BamIII primer (Rv) sequence
cgatgccccacaggaatcgataggggtttttgcggg

SEQ ID NO: 67 - DNA, 35 bp, Artificial
  Description of Artificial sequence mouse ASCT2 NcoI primer (Fw) sequence
cgggagatcaattcaacgatggtccagcttctctg

SEQ ID NO: 68 - DNA, 35 bp, Artificial
  Description of Artificial sequence mouse ASCT2 NcoI primer (Rv) sequence
cagagaagctggaccatcgttgaattgatctcccg

SEQ ID NO: 69 - DNA, 30 bp, Artificial
  Description of Artificial sequence human ASCT2 EL4 primer (Fw) sequence
gtgcgtggaggagaagaatggcgtggccaa

SEQ ID NO: 70 - DNA, 30 bp, Artificial
  Description of Artificial sequence human ASCT2 EL4 primer (Rv) sequence
ttggccacgccattcttctcctccacgcac

SEQ ID NO: 71 - protein, 120 aa, Artificial
  PEPTIDE (1)..(120): Description of Artificial sequence KM4008 HV0 sequence
QVQLVQSGSELKKPGAPVKVSCKASGYTFSTFGMSWVRQAPGQGLEWMGWIHTYAGVPIYGDDFKGRFVFSLDTSVSTAYLQISSLKAEDTAVYYCARRSDNYRYFFDYWGQGTTVTVSS

SEQ ID NO: 72 - protein, 107 aa, Artificial
  PEPTIDE (1)..(107): Description of Artificial sequence KM4008 LV0 sequence
DIQMTQSPSSLSASVGDRVTITCRASQDIRNYLNWYQQKPGKAPKLLIYYTSRLHSGVPSRFSGSGSGTDFTLTISSLQPEDFATYYCQQGHTLPPTFGQGTKLEIK

SEQ ID NO: 73 - DNA, 360 bp, Artificial
  CDS (1)..(360): Description of Artificial sequence KM4008 HV0 sequence
caggtgcagttggtccagtccggctctgagctgaagaagcctggtgcccctgtgaaggtctcctgcaaggcttctggttacaccttcagcacctttggcatgagctgggtgcgacaggcccctggtcaagggcttgagtggatgggttggattcacacctacgctggtgtgccaatctatggtgatgacttcaagggtcgattcgtcttcagcctggacacatccgtgagcacagcctacttgcagatcagcagcctgaaggctgaggacacggccgtgtattactgtgcgcgccgttcggataattaccgttatttttttgactattggggccagggtaccacagtcaccgtctcctca

SEQ ID NO: 74 - DNA, 321 bp, Artificial
  CDS (1)..(321): Description of Artificial sequence KM4008 LV0 sequence
gacatccagatgacccagtctccttcctcactgtctgcatctgtaggtgaccgagtcaccatcacttgccgggcaagtcaggacattcggaattatttaaactggtatcagcagaaaccagggaaagcccctaagctcctgatctattacacatcacgattacactcaggggtcccatcacggttcagcggcagtggttctgggacagatttcactctcaccatcagcagtctgcagcctgaagatttcgcaacttattactgccaacagggtcatacgcttcctccgacgttcggccaagggaccaagctggagatcaaa

SEQ ID NO: 75 - DNA, 360 bp, Artificial
  CDS (1)..(360): Description of Artificial sequence HV2 sequence
caggtgcagttggtccagtccggctctgagctgaagaagcctggtgcccctgtgaaggtctcctgcaaggcttctggttacaccttcagcacctttggcatgagctgggtgcgacaggcccctggtcaagggcttaagtggatgggttggattcacacctacgctggtgtgccaatctatggtgatgacttcaagggtcgattcgtcttcagcctggacacatccgtgagcacagcctacttgcagatcagcagcctgaaggctgaggacacggccgtgtatttctgtgcgcgccgttcggataattaccgttatttttttgactattggggccagggtaccacagtcaccgtctcctca

SEQ ID NO: 76 - protein, 120 aa, Artificial
  PEPTIDE (1)..(120): Description of Artificial sequence HV2 sequence
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 77 - DNA, 360 bp, Artificial
  CDS (1)..(360): Description of Artificial sequence HV4 sequence
caggtgcagttggtccagtccggctctgagctgaagaagcctggtgcccctgtgaagatctcctgcaaggcttctggttacaccttcagcacctttggcatgagctgggtgcgacaggcccctggtcaagggcttaagtggatgggttggattcacacctacgctggtgtgccaatctatggtgatgacttcaagggtcgattcgtcttcagcctggacacatccgtgagcacagcctacttgcagatcagcagcctgaaggctgaggacacggccgtgtatttctgtgcgcgccgttcggataattaccgttatttttttgactattggggccagggtaccacactcaccgtctcctca

SEQ ID NO: 78 - protein, 120 aa, Artificial
  PEPTIDE (1)..(120): Description of Artificial sequence HV4 sequence
QVQLVQSGSELKKPGAPVKISCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTLTVSS

SEQ ID NO: 79 - DNA, 360 bp, Artificial
  CDS (1)..(360): Description of Artificial sequence HV7 sequence
caggtgcagttggtccagtccggccctgagctgaagaagcctggtgcccctgtgaagatctcctgcaaggcttctggttacaccttcagcacctttggcatgagctgggtgaaacaggcccctggtcaagggcttaagtggatgggttggattcacacctacgctggtgtgccaatctatggtgatgacttcaagggtcgattcgtcttcagcctggacacatccgtgagcacagcctacttgcagatcagcagcctgaaggctgaggacacggccacatatttctgtgcgcgccgttcggataattaccgttatttttttgactattggggccagggtaccacactcaccgtctcctca

SEQ ID NO: 80 - protein, 120 aa, Artificial
  PEPTIDE (1)..(120): Description of Artificial sequence HV7 sequence
QVQLVQSGPELKKPGAPVKISCKASGYTFSTFGMSWVKQAPGQGLKWMGWIHTYAGVPIYGDDFKGRFVFSLDTSVSTAYLQISSLKAEDTATYFCARRSDNYRYFFDYWGQGTTLTVSS

SEQ ID NO: 81 - DNA, 360 bp, Artificial
  CDS (1)..(360): Description of Artificial sequence HV10 sequence
cagatccagttggtccagtccggccctgagctgaagaagcctggtgcccctgtgaagatctcctgcaaggcttctggttacaccttcacaacctttggcatgagctgggtgaaacaggcccctggtcaagggcttaagtggatgggttggattcacacctacgctggtgtgccaatctatggtgatgacttcaagggtcgattcgtcttcagcctggacacatccgtgagcacagcctacttgcagatcagcagcgtcaaggctgaggacacggccacatatttctgtgcgcgccgttcggataattaccgttatttttttgactattggggccagggtaccacactcaccgtctcctca

SEQ ID NO: 82 - protein, 120 aa, Artificial
  PEPTIDE (1)..(120): Description of Artificial sequence HV10 sequence
QIQLVQSGPELKKPGAPVKISCKASGYTFTTFGMSWVKQAPGQGLKWMGWIHTYAGVPIYGDDFKGRFVFSLDTSVSTAYLQISSVKAEDTATYFCARRSDNYRYFFDYWGQGTTLTVSS

SEQ ID NO: 83 - DNA, 321 bp, Artificial
  CDS (1)..(321): Description of Artificial sequence LV3 sequence
gacatccagatgacccagtctccttcctcactgtctgcatctctgggtgaccgagtcaccatcacttgccgggcaagtcaggacattcggaattatttaaactggtatcagcagaaaccagggaaagcccctaagctcctgatctattacacatcacgattacactcaggggtcccatcacggttcagcggcagtggttctgggacagattatactctcaccatcagcagtctgcagcctgaagatttcgcaacttatttttgccaacagggtcatacgcttcctccgacgttcggccaagggaccaagctggagatcaaa

SEQ ID NO: 84 - protein, 107 aa, Artificial
  PEPTIDE (1)..(107): Description of Artificial sequence LV3 sequence
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 85 - DNA, 2758 bp, Mus musculus
  CDS (556)..(2223): mouse ASCT2 nucleotide sequence GenBank Accession number NM_009201
ggtcgcttccgtctagcagcctcagagacacgcggcgcggcctgggaggtttctccttggcgccactttccatatcccccgggctgcttcctatttgcatctggaaatttgatttttctctccagagcccgaaacgctcggtccactccttctagggctccagtacccaagagtccgagagccaggcgtcctaatcctgggatctcggtctcccggagcaaggcatcccggatctatgccacttgggttgagcttcctgaagacctcagctacctgagtttccttactccttcagggagtcaaagaaccctctgaagagctgttccctactccaacaaggcgctctcaactcccagttttcggggattcgacatcaccaggaaaaataaagggatctccgcatacagctcagggagcctaggaccctccagtttccgagatccggtggacgcataacttcaaggaccgctgcaaagtttcagcctccattctcggtcctaaggacgtcacacctagtctccaggctcacaaggaacctccctgttacggctcagcatggcagtggatccccctaaggctgaccccaaaggggtagtagcggtggattccaccgcgaacggtggccccgcgctgggatccagagaggaccagagtgcgaaagcaggtggttgctgcggttcccgtgaccgggtgcgccgctgcattcgcgccaacctgctggtgctgctcacggtggctgcggtggtggctggcgtggggctggggctgggggtctcggcggcgggcggtgctgacgcgctgggtcccgcgcgcttgaccgctttcgccttcccgggagagctgctgctgcgtctgctgaagatgatcatcctgccgctcgtggtgtgcagcctgatcggaggtgcagccagcttggaccctagcgcgctcggtcgtgtgggcgcctgggcgctgctctttttcctggtcaccacactgctcgcgtcggcgctcggcgtgggtttggccctggcgctgaagccgggcgccgccgttaccgccatcacctccatcaacgactctgttgtagacccctgtgcccgcagtgcaccaaccaaagaggtgctggattcctttctagatctcgtgaggaatattttcccctccaatctggtgtctgctgccttccgctcttttgctacctcatatgaacccaaagacaactcatgtaaaataccgcaatcctgtatccagcgggagatcaattcaaccatggtccagcttctctgtgaggtggagggaatgaacatcctgggcctggtggtcttcgctatcgtctttggtgtggctctgcggaagctggggcccgagggtgagctgctcattcgtttcttcaactccttcaatgatgccaccatggtcctggtctcctggattatgtggtacgcacccgttggaatcctgttcctggtggccagcaagattgtggagatgaaagacgtccgccagctcttcatcagcctcggcaaatacattctgtgctgcctgctgggccacgccatccacgggctcctggttctgcctctcatctacttcctcttcacccgcaaaaatccctatcgattcctgtggggcatcatgacacccctggccactgctttcgggacctcttctagctctgccaccttgcctctgatgatgaagtgtgtagaggagaagaatggtgtggccaaacacatcagccggttcatcctacccatcggcgccacggtcaacatggacggggcggcgctgttccagtgtgtggcggcagtgttcatcgcacaactaaacggggtgtccctggacttcgtgaagatcatcaccatcctggtcacagccacagcatccagtgtcggtgcggcagggatccctgcagggggcgtcctcaccctcgccatcatcctggaagcagtcagcctgcctgtgaaggacatctccttgatcttggccgtggactggctagtggacaggtcctgtactgtcctcaacgtggaaggtgatgcttttggggctggactgcttcagagttacgtggatcgaaccaagatgccgagctcagagcccgaattgatccaggtgaagaacgaggtgtctctgaatccactgcccctcgccacagaggaggggaatcccctcctgaaacagtaccagggacccaccggggactccagtgccacgttcgaaaaggaatctgtcatgtgaatatcaggagggactttctacgccctggtgacttcacgttggaagccgggtcttgcgagctggagaaatggactggatgagggctctaggggtgggctgtttacacactccaggaatctaggggtcggtgtctccttcctggagaggctattaggagccttgttgctggggtgcatatgcttatatgtgaatgtgtccacaaatcccaccccctctccgttctgtcccccattcaaggaatgacattctcagtcacccagagccatctggcctaccctctcaggaaatgggttgtcctgtagagttctctacccttttaaaggcaaaagtattgggatgcaatactatcttaatgtccctatctgtggtgtgtgtgcacttgctgtgaccttcccagtagtccctccatgacagaaaacactcccatgagtctcgagggaaagccaagaataaatgtcactccagaacacatttttttttagcaataaaatggtgtcaaatgtattgaccatggccctagagtctgagtaatacc

SEQ ID NO: 86 - protein, 555 aa, Mus musculus
  PEPTIDE (1)..(555): ASCT2 peptide sequence GenPept Accession number NP_033227
MAVDPPKADPKGVVAVDSTANGGPALGSREDQSAKAGGCCGSRDRVRRCIRANLLVLLTVAAVVAGVGLGLGVSAAGGADALGPARLTAFAFPGELLLRLLKMIILPLVVCSLIGGAASLDPSALGRVGAWALLFFLVTTLLASALGVGLALALKPGAAVTAITSINDSVVDPCARSAPTKEVLDSFLDLVRNIFPSNLVSAAFRSFATSYEPKDNSCKIPQSCIQREINSTMVQLLCEVEGMNILGLVVFAIVFGVALRKLGPEGELLIRFFNSFNDATMVLVSWIMWYAPVGILFLVASKIVEMKDVRQLFISLGKYILCCLLGHAIHGLLVLPLIYFLFTRKNPYRFLWGIMTPLATAFGTSSSSATLPLMMKCVEEKNGVAKHISRFILPIGATVNMDGAALFQCVAAVFIAQLNGVSLDFVKIITILVTATASSVGAAGIPAGGVLTLAIILEAVSLPVKDISLILAVDWLVDRSCTVLNVEGDAFGAGLLQSYVDRTKMPSSEPELIQVKNEVSLNPLPLATEEGNPLLKQYQGPTGDSSATFEKESVM

SEQ ID NO: 87 - DNA, 450 bp, Artificial
  Description of Artificial sequence HV2 synthetic gene sequence
gcggccgcgacccctcaccatgagagtgcttattttattgtggctgttcacagcctttcctggtattcttagtcaggttcaattagtccagtccggctctgagctgaagaagcctggtgcccctgtgaaggtctcctgcaaggcttctggttacaccttcagcacctttggcatgagctgggttcgtcaagcccctggtcaagggcttaagtggatgggttggattcacacctacgctggtgttcctatctatggcgatgatttcaagggtcgattcgtcttcagcctggacacatccgtgagcacagcctacttgcagatcagcagcctgaaggctgaggacacggccgtgtatttctgtgcgcgccgttcggataattaccgttatttttttgactattggggtcaaggcaccacagtcactgtttcatcagcctccaccaagggccc

SEQ ID NO: 88 - DNA, 410 bp, Artificial
  Description of Artificial sequence LV3 synthetic gene sequence
gaattcgcctcctcaaaatgcattttcaagtgcagattttcagcttcctgcttatttcggcctcagtcataatgtccagaggagacatccagatgacccagtctccttcctcactgtctgcatctctgggtgaccgagtcaccatcacttgccgggcatctcaagatattcggaattatttaaactggtatcagcagaaaccagggaaagcccctaagctccttatttattacacatcacgattacactcaggggtcccatcacggttcagcggcagtggttctgggacagattatactctcaccatcagcagtctgcagcctgaagatttcgcaacttatttctgtcaacaaggtcatacgcttcctccgacgttcggccaagggaccaagctggagatcaaacgtacg

SEQ ID NO: 89 - DNA, 450 bp, Artificial
  Description of Artificial sequence HV10 synthetic gene sequence
gcggccgcgacccctcaccatgagagtgcttattttattgtggctgttcacagcctttcctggtattcttagtcagatccagttggtccagtccggccctgagctgaagaagcctggtgcccctgtgaagatctcctgcaaggcttctggttacaccttcacaacctttggcatgagctgggttaaacaagcccctggtcaagggcttaagtggatgggttggattcacacctacgctggtgttcctatctatggcgatgatttcaagggtcgattcgtcttcagcctggacacatccgtgagcacagcctacttgcagatcagcagcgtcaaggctgaggacacggccacatatttctgtgcgcgccgttcggataattaccgttatttttttgactattggggtcaaggcaccacactcactgtttcatcagcctccaccaagggccc

(19) United States
(12) Patent Application Publication
(10) Pub. No.: US 2014/0134730 A1
(43) Pub. Date: May 15, 2014

(54) METHOD FOR INHIBITING CELL PROLIFERATION BY OXIDATIVE STRESS

(71) Applicant: KYOWA HAKKO KIRIN CO., LTD, Tokyo (JP)

(72) Inventors: Aya SASAKAWA, Tokyo (JP); Yoshiki SUMITOMO, Tokyo (JP)

(73) Assignee: KYOWA HAKKO KIRIN CO., LTD, Tokyo (JP)

(21) Appl. No.: 14/062,009

(22) Filed: Oct. 24, 2013

Related U.S. Application Data
(60) Provisional application No. 61/717,703, filed on Oct. 24, 2012.

(51) Int. Cl. C07K 16/30

(52) U.S. Cl. CPC C07K 16/30
USPC 435/375; 530/388.85

(57) ABSTRACT

The present invention relates to an intracellular oxidative stress promoter, comprising a monoclonal antibody having an inhibitory activity of intracellular uptake of neutral amino acids or an antibody fragment thereof as an active ingredient. Also, the present invention relates to a cell proliferation inhibitor, comprising a monoclonal antibody having an inhibitory activity of intracellular uptake of neutral amino acids or an antibody fragment thereof as an active ingredient and promoting intracellular oxidative stress.

Further, the present invention relates to a method for promoting intracellular oxidative stress, comprising administration of a monoclonal antibody having an inhibitory activity of intracellular uptake of neutral amino acids or an antibody fragment thereof. Also, the present invention relates to a method for inhibiting cell proliferation, comprising administration of a monoclonal antibody having an inhibitory activity of intracellular uptake of neutral amino acids or an antibody fragment thereof and promotion of intracellular oxidative stress.

### BACKGROUND OF THE INVENTION

1. Field of the Invention

The present invention relates to an intracellular oxidative stress promoter, comprising a monoclonal antibody having an inhibitory activity of intracellular uptake of neutral amino acids or an antibody fragment thereof as an active ingredient. Also, the present invention relates to a cell proliferation inhibitor, comprising a monoclonal antibody having an inhibitory activity of intracellular uptake of neutral amino acids or an antibody fragment thereof as an active ingredient and promoting intracellular oxidative stress.

Further, the present invention relates to a method for promoting intracellular oxidative stress, comprising administration of a monoclonal antibody having an inhibitory activity of intracellular uptake of neutral amino acids or an antibody fragment thereof. Also, the present invention relates to a method for inhibiting cell proliferation, comprising administration of a monoclonal antibody having an inhibitory activity of intracellular uptake of neutral amino acids or an antibody fragment thereof and promotion of intracellular oxidative stress.

2. Brief Description of the Background Art

An ASCT2 polypeptide is a 10-times transmembrane protein consisting of 541 amino acids in full-length form, and functions as a transporter which transports neutral amino acids through a cell membrane depending on a sodium ion. As another name, Solute carrier family 1 (neutral amino acid transport), member 5, Solute carrier family 1 member 5, or SLC1A5 is used. With respect to the link between the development of cancer and the expression of ASCT2 or SLC1A5, it has been known that expression levels of three SLC1A5, SLC7A5 and SLC38A5 are significantly increased in cancerous tissues by studies using expressed sequence tag (EST) database (Non-Patent Literature 1).

Further, it has been found that the expression of SLC1A5 is not recognized in a normal liver, but is recognized in clinical tissues of hepatocellular carcinoma or hepatoblastoma (Non-Patent Literature 2). In addition, it has been found that the expression of SLC1A5 is higher in clinical tissues of poorly-differentiated astrocytoma or glioblastoma multiforme than that of normal tissues (Non-Patent Literature 3).

Further, the expression of ASCT2 is detected in clinical tissues of colorectal cancer and prostate cancer by immunohistological staining or Western blotting, and patients who highly express ASCT2 have a poor prognosis (Non-Patent Literature 4).

Furthermore, it has been found that ASCT2 is responsible for the uptake of glutamine in several cell lines of colorectal cancer, liver cancer, breast cancer, astrocytoma and neuroblastoma (Non-Patent Literatures 5, 6, 7 and 8), and that the proliferation of cells is suppressed by competitively inhibiting intracellular uptake of glutamine using an alanine-serine-threonine mixture which is a substrate for ASCT2 (Non-Patent Literature 9).

Further, although it has been reported that intracellular uptake of amino acids is inhibited and cell proliferation is suppressed by an anti-ASCT2 antibody (Patent Literatures 1 and 2), their mechanisms have not been clarified.

### PRIOR ART DOCUMENTS

### Patent Literature

[Patent Literature 1] WO 2010/008075
[Patent Literature 2] WO 2011/087091

### Non-Patent Literature

[Non-Patent Literature 1] Seminars in Cancer Biology, 15, 254 (2005)
[Non-Patent Literature 2] Am. J. Physiol. Gastrointest. Liver Physiol., 283, G1062 (2002)
[Non-Patent Literature 3] NeuroReport, 15, 575 (2004)
[Non-Patent Literature 4] Anticancer Research, 22, 2555 (2002), Anticancer Research, 23, 3413 (2003)
[Non-Patent Literature 5] 1 J. Surgical Research, 90, 149 (2000)
[Non-Patent Literature 6] J. Cellular Physiology, 176, 166 (1998)
[Non-Patent Literature 7] J. Neuroscience Research, 66, 959 (2001)
[Non-Patent Literature 8] Am. J. Physiol. Cell Physiol., 282, C1246 (2002)
[Non-Patent Literature 9] J. Surgical Research, 90, 149 (2000)

### SUMMARY OF INVENTION

The present invention aims to provide an intracellular oxidative stress promoter, comprising a monoclonal antibody having an inhibitory activity of intracellular uptake of neutral amino acids or an antibody fragment thereof as an active ingredient. Also, the present invention aims to provide a cell proliferation inhibitor, comprising a monoclonal antibody having an inhibitory activity of intracellular uptake of neutral amino acids or an antibody fragment thereof as an active ingredient and promoting intracellular oxidative stress.

Further, the present invention aims to provide a method for promoting intracellular oxidative stress, comprising administration of a monoclonal antibody having an inhibitory activity of intracellular uptake of neutral amino acids or an antibody fragment thereof. Also, the present invention aims to provide a method for inhibiting cell proliferation, comprising administration of a monoclonal antibody having an inhibitory activity of intracellular uptake of neutral amino acids or an antibody fragment thereof and promotion of intracellular oxidative stress.

The intracellular oxidative stress promoter, the cell proliferation inhibitor, the method for promoting intracellular oxidative stress, and the method for inhibiting cell proliferation of the present invention utilize a monoclonal antibody having an inhibitory activity of intracellular uptake of neutral amino acids or an antibody fragment thereof as an active ingredient, so that intracellular oxidative stress is promoted, uptake of neutral amino acids is inhibited to suppress ATP production, DNA double strand damage or cell cycle arrest in the G1 phase are caused, and inhibition of cell proliferation or cell death can be caused.

### BRIEF DESCRIPTION OF THE DRAWINGS

FIG. 1 shows changes in the intracellular metabolic response to KM4008HV2LV3 in SNU-16 cancer cell line. Among the results of metabolome analysis by CE-TOFMS in KM4008HV2LV3-treated or solvent-treated cells, the relative areas of the intermediate metabolites of the glutathione synthesis system and TCA cycle system at 2 hours or 8 hours after antibody addition are shown. The horizontal axis represents the time after KM4008HV2LV3 or solvent treatment, and the vertical axis represents the relative area. In the graph, the black color represents the solvent-treated group, and the white color represents the KM4008HV2LV3-treated group.

FIG. 2 shows the result of antibody array involved in oxidative stress. At 24 hours after addition of a drug to SNU-16 cells, the cells were disrupted and hybridized with the antibody array of Full Moon Biosystems, followed by detection of signal changes. Among the antibodies which exhibited 2-fold or more of difference in the signal intensity between the solvent-treated cells and KM4008HV2LV3-treated cells at 24 hours after solvent or antibody addition, the results of the antibodies involved in oxidative stress are shown. The horizontal axis represents the cultivation period (hrs) after antibody addition, and the vertical axis represents the value obtained by dividing the signal value of KM4008HV2LV3-treated cells by the signal value of solvent-treated cells.

FIG. 3 shows the result of antibody array involved in the response to double strand break. At 24 hours after addition of the drug to SNU-16 cells, the cells were disrupted and hybridized with the antibody array of Full Moon Biosystems, followed by detection of signal changes. Among the antibodies which exhibited 2-fold or more of difference in signal intensity between the solvent-treated cells and KM4008HV2LV3-treated cells at 24 hours after solvent or antibody addition, the results of the antibodies involved in the response to double strand break are shown. The horizontal axis represents the cultivation period (hrs) after antibody addition, and the vertical axis represents the value obtained by dividing the signal value of KM4008HV2LV3-treated cells by the signal value of solvent-treated cells.

FIG. 4 shows the cell cycle changes that are caused by KM4008HV2LV3. At 24 hours after addition of the drug to SNU-16 cells, the cells were fixed, and stained with propidium iodide, and then the fluorescence intensity was measured by a flow cytometer, and thus obtained histogram is shown. The horizontal axis represents FL2 fluorescence intensity indicating the DNA content. In the vertical axis, the relative cell number (%) represents the relative number of cells which is obtained by assuming the number of peak cells in each treatment group as 100%. NT indicates solvent treatment, Ab indicates KM4008HV2LV3 (10 μg/mL) treatment, and H₂O₂ indicates hydrogen peroxide (50 μM) treatment.

FIG. 5 shows analysis of the cell death that is caused by KM4008HV2LV3. At 72 hours after addition of the drug to SNU-16 cells, cells were stained with annexin V-FITC, and then the fluorescence intensity was measured by a flow cytometer, and thus obtained histogram is shown. The horizontal axis represents FL1 fluorescence intensity indicating the fluorescence intensity of annexinV-FITC. In the vertical axis, the relative cell number (%) represents the relative number of cells calculated by assuming the number of peak cells in each treatment group as 100%. NT indicates solvent treatment, KM4008HV2LV3 indicates KM4008HV2LV3 (10 μg/mL) treatment, and H₂O₂ indicates hydrogen peroxide (50 μM) treatment.

FIG. 6 shows recovery of KM4008HV2LV3-caused cell proliferation inhibition by antioxidant. At 48 hours or 72 hours after addition of antibody and N-acetylcysteine (NAC) to SNU-16 cells, PI staining was performed, and the number of live cells was measured using a flow cytometer, and thus obtained result is shown. The horizontal axis represents the cultivation period (hrs) after antibody addition, and the vertical axis represents the number of live cells (cells/mL). n=3, error bars represent standard deviations. NT indicates solvent treatment, Ab indicates KM4008HV2LV3 treatment, and NAC indicates NAC treatment.

### DETAILED DESCRIPTION OF THE INVENTION

This application relates to the inventions (1) to (24).

(1) An intracellular oxidative stress promoter, comprising a monoclonal antibody having an inhibitory activity of intracellular uptake of neutral amino acids or an antibody fragment thereof as an active ingredient.
(2) The intracellular oxidative stress promoter according to (1), wherein the neutral amino acid is glutamine.
(3) The intracellular oxidative stress promoter according to (1), wherein the monoclonal antibody is an antibody which binds to the extracellular region of a neutral amino acid transporter.
(4) The intracellular oxidative stress promoter according to (1), wherein the monoclonal antibody is an antibody which binds to the extracellular region of a system ASC amino acid transporter 2 (ASCT2).
(5) The intracellular oxidative stress promoter according to (1), wherein the monoclonal antibody is an antibody selected from (A) to (C):

(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;

(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;

(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

(6) The intracellular oxidative stress promoter according to (1), wherein the cell is a cancer cell.
(7) A cell proliferation inhibitor, comprising a monoclonal antibody having an inhibitory activity of intracellular uptake of neutral amino acids or an antibody fragment thereof as an active ingredient and promoting intracellular oxidative stress.
(8) The cell proliferation inhibitor according to (7), wherein the neutral amino acid is glutamine.
(9) The cell proliferation inhibitor according to (7), wherein the monoclonal antibody is an antibody which binds to the extracellular region of a neutral amino acid transporter.
(10) The cell proliferation inhibitor according to (7), wherein the monoclonal antibody is an antibody which binds to the extracellular region of ASCT2.
(11) The cell proliferation inhibitor according to (7), wherein the monoclonal antibody is an antibody selected from (A) to (C):

(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;

(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;

(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

(12) The cell proliferation inhibitor according to (7), wherein the cell is a cancer cell.
(13) A method for promoting intracellular oxidative stress, comprising administration of a monoclonal antibody having an inhibitory activity of intracellular uptake of neutral amino acids or an antibody fragment thereof.
(14) The method according to (13), wherein the neutral amino acid is glutamine.
(15) The method according to (13), wherein the monoclonal antibody is an antibody which binds to the extracellular region of a neutral amino acid transporter.
(16) The method according to (13), wherein the monoclonal antibody is an antibody which binds to the extracellular region of a system ASC amino acid transporter 2 (ASCT2).
(17) The method according to (13), wherein the monoclonal antibody is an antibody selected from (A) to (C):

(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;

(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;

(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

(18) The method according to (13), wherein the cell is a cancer cell.
(19) A method for inhibiting cell proliferation, comprising administration of a monoclonal antibody having an inhibitory activity of intracellular uptake of neutral amino acids or an antibody fragment thereof and promotion of intracellular oxidative stress.
(20) The method according to (19), wherein the neutral amino acid is glutamine.
(21) The method according to (19), wherein the monoclonal antibody is an antibody which binds to the extracellular region of a neutral amino acid transporter.
(22) The method according to (19), wherein the monoclonal antibody is an antibody which binds to the extracellular region of ASCT2.
(23) The method according to (19), wherein the monoclonal antibody is an antibody selected from (A) to (C):

(A) a monoclonal antibody in which CDR1, CDR2 and CDR3 of YH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;

(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;

(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

(24) The method according to (19), wherein the cell is a cancer cell.

The present invention relates to an intracellular oxidative stress promoter, comprising a monoclonal antibody having an inhibitory activity of intracellular uptake of neutral amino acids or an antibody fragment thereof as an active ingredient.

Also, the present invention relates to a cell proliferation inhibitor, comprising a monoclonal antibody having an inhibitory activity of intracellular uptake of neutral amino acids or an antibody fragment thereof as an active ingredient and promoting intracellular oxidative stress.

The present invention includes a tricarboxylic acid (TCA) cycle (also described as citric-acid cycle) inhibitor, which includes a monoclonal antibody having an inhibitory activity of intracellular uptake of neutral amino acids or an antibody fragment thereof as an active ingredient.

Further, the present invention includes a cell proliferation inhibitor, comprising a monoclonal antibody having an inhibitory activity of intracellular uptake of neutral amino acids or an antibody fragment thereof as an active ingredient and inhibiting TCA cycle.

In the present invention, examples of the neutral amino acids may include glutamine, alanine, serine and threonine, and preferably, glutamine.

In the present invention, a route of the intracellular uptake of neutral amino acids is not particularly limited. Preferably, the intracellular uptake may be mediated by a neutral amino acid transporter, and more preferably, the intracellular uptake may be mediated by a system ASC amino acid transporter 2 (ASCT2).

As ASCT2 of the present invention, species is not particularly limited. Preferably, it may be ASCT2 derived from a mammal, and specifically ASCT2 derived from a human.

The amino acid sequence information of ASCT2 is available from a known database such as NCBI (http://www.ncbi.nlm.nih.gov/). Examples include human ASCT2 amino acid sequence having the amino acid sequence shown in SEQ ID NO:2 (NCBI Accession No. NP 005619), mouse ASCT2 amino acid sequence having the amino acid sequence shown in SEQ ID NO:86 (NCBI Accession No. NP 033227), and the like.

Examples of ASCT2 in the present invention include a polypeptide having the amino acid sequence shown in SEQ ID NO:2 or a polypeptide consisting of an amino acid sequence wherein one or more amino acids are deleted, substituted or added in the amino acid sequence shown in SEQ ID NO:2 and having a function of ASCT2. ASCT2 in the present invention also includes a polypeptide having 60% or more homology, preferably 80% or more homology, further preferably 90% or more homology, the most preferably 95% or more homology to the amino acid sequence shown in SEQ ID NO:2 and having a function of ASCT2.

The polypeptide having an amino acid sequence wherein one or more amino acids are deleted, substituted or added in the amino acid sequence shown in SEQ ID NO:2 can be obtained by using site-specific mutagenesis [Molecular Cloning, A Laboratory Manual, Second Edition, Cold Spring Harbor Laboratory Press (1989), Current Protocols in Molecular Biology, John Wiley & Sons (1987-1997), Nucleic Acids Research, 10, 6487 (1982), Proc. Natl. Acad. Sci., USA, 79, 6409 (1982), Gene, 34, 315 (1985), Nucleic Acids Research, 13, 4431 (1985), Proc. Natl. Acad. Sci. USA, 82, 488 (1985), etc.]. For example, it can be obtained by introducing a site-specific mutation into DNA having the nucleotide sequence encoding a polypeptide having the amino acid sequence shown in SEQ ID NO:2. The number of amino acids which are deleted, substituted or added is not specifically limited. The number is preferably 1 to dozens, such as 1 to 20, more preferably 1 to several, such as 1 to 5.

Examples of a gene encoding ASCT2 include DNA consisting of the nucleotide sequence shown in SEQ ID NO:1. The gene encoding ASCT2 in the present invention also includes a gene comprising a DNA consisting of a nucleotide sequence in which one or more bases are deleted, substituted or added in the nucleotide sequence shown in SEQ ID NO:1 and encoding a polypeptide having the function of ASCT2; a gene comprising a DNA consisting of a nucleotide sequence having at least 60% or more homology, preferably 80% or more homology, and further preferably 95% or more homology with the nucleotide sequence shown in SEQ ID NO:1 and encoding a polypeptide having the function of ASCT2; a gene consisting of DNA which hybridizes with a DNA having the nucleotide sequence shown in SEQ ID NO:1 under stringent conditions and comprising a DNA encoding a polypeptide having the function of ASCT2; and the like.

The DNA which hybridizes under stringent conditions refers to a DNA which is obtained by colony hybridization, plaque hybridization, Southern hybridization, DNA microarray, or the like using a DNA having the nucleotide sequence shown in SEQ ID NO:1 as a probe and which is capable of hybridization.

A specific example of such DNA is a DNA which can be identified by the hybridization using the filter or slide glass on which a DNA derived from hybridized colony or plaque, or PCR product or oligo DNA having said sequence, under the presence of 0.7 to 1.0 mol/L sodium chloride at 65° C. [Molecular Cloning, A Laboratory Manual, Second Edition, Cold Spring Harbor Lab. Press (1989), Current Protocols in Molecular Biology, John Wiley & Sons (1987-1997); DNA Cloning 1: Core Techniques, A Practical Approach, Second Edition, Oxford University (1995)], and then washing the filter or slide glass at 65° C. with a 0.1 to 2-fold concentration SSC solution (1-fold concentration SSC solution: 150 mmol/l sodium chloride and 15 mmol/l sodium citrate).

The DNA capable of hybridization includes DNA having at least 60% or more homology, preferably 80% or more homology, further preferably 95% or more homology to the nucleotide sequence shown in SEQ ID NO:1.

In the nucleotide sequence of the gene encoding a protein of a eukaryote, genetic polymorphism is often found. The ASCT2 gene in the present invention also includes a gene which is used in the present invention and in which minor mutations are generated in the nucleotide sequence by such polymorphism.

The value of the homology described in the present invention may be a value calculated by using a homology search program known by the skilled person, unless specifically indicated. Regarding the nucleotide sequence, the value may be calculated by BLAST [J. Mol. Biol., 215, 403 (1990)] with a default parameter or the like. Further, regarding the amino acid sequence, the value may be calculated by using BLAST2 [Nucleic Acids Res., 25, 3389 (1997); Genome Res., 7, 649 (1997); http://www.ncbi.nlm.nih.gov/Education/BLASTinfo/information3.html] with a default parameter or the like.

As the default parameter, G (cost to open gap) is 5 for the nucleotide sequence and 11 for the amino acid sequence; −E (cost to extend gap) is 2 for the nucleotide sequence and 1 for the amino acid sequence; −q (penalty for nucleotide mismatch) is −3; −r (reward for nucleotide match) is 1; −e (expect value) is 10; −W (wordsize) is 11 residues for the nucleotide sequence and 3 residues for the amino acid sequence; −y (dropoff (X) for blast extensions in bits) is 20 for blastn and 7 for a program other than blastn; −X (X dropoff value for gapped alignment in bits) is 15; and −Z (final X dropoff value for gapped alignment in bits) is 50 for blastn and 25 for a program other than blastn (http://www.ncbi.nlm.nih.gov/blast/html/blastcgihelp.html).

A polypeptide consisting of a partial sequence of the amino acid sequence shown in SEQ ID NO:2 can be prepared by methods known to those skilled in the art. For example, such polypeptide can be prepared by partially deleting a DNA encoding the amino acid sequence shown in SEQ ID NO:2, and culturing a transformant into which an expression vector containing the partially deleted DNA is introduced. Based on the thus constructed polypeptide or DNA, a polypeptide in which one or more amino acids are deleted, substituted or added in a partial sequence of the amino acid sequence shown in SEQ ID NO:2 can also be obtained, in accordance with the same method as described above.

Further, the polypeptide consisting of a partial sequence of the amino acid sequence shown in SEQ ID NO:2, or the polypeptide in which one or more amino acids are deleted, substituted or added in a partial sequence of the amino acid sequence shown in SEQ ID NO:2 can also be produced by a chemical synthesis method such as a fluorenylmethyloxycarbonyl (Fmoc) method, a t-butyloxycarbonyl (tBoc) method, or the like.

The monoclonal antibody of the present invention may be an antibody which binds to the extracellular region of the neutral amino acid transporter, and preferably, an antibody which binds to the extracellular region of ASCT2.

The extracellular region of the neutral amino acid transporter in the present invention may be a region predicted by a known transmembrane region prediction programs SOSUI (http://sosui.proteome.bio.tuat.ac.jp/sosuiframe0.html), TMHMM ver. 2 (http://www.cbs.dtu.dk/services/TMHMM-2.0/), ExPASy Proteomics Server (http://Ca.expasy.org/) or the like, based on the amino acid sequence of the transporter.

Specifically, examples of the extracellular region of ASCT2 in the present invention include a region predicted by a known transmembrane region prediction programs SOSUI (http://sosui.proteome.bio.tuat.ac.jp/sosuiframe0.html), TMHMM ver. 2 (http://www.cbs.dtu.dk/services/TMHMM-2.0/), ExPAsy Proteomics Server (http://Ca.expasy.org/), or the like, based on the amino acid sequence of the polypeptide shown in SEQ ID NO:2.

More specifically, examples include five regions, at positions 74 to 98, at positions 154 to 224, at positions 287 to 305, at positions 357 to 376 and at positions 420 to 425 which are the extracellular domain predicted by ExPASy Proteomics Server.

Alternatively, examples include five regions, at positions 65 to 88, at positions 152 to 224, at positions 288 to 306, at positions 361 to 380 and at positions 447 to 451 which are the extracellular domain predicted in the literature [J. Biol. Chemistry, 271, 18657 (1996)] or [J. Virology, 73, 4470 (1999)].

In the present invention, the five extracellular regions of ASCT2 are indicated as an EL1 region, an EL2 region, an EL3 region, an EL4 region and an EL5 region sequentially from the N-terminal side. For example, the EL2 region in an amino acid sequence of the ASCT2 polypeptide shown in SEQ ID NO:2 corresponds to positions 154 to 224 or positions 152 to 224.

The monoclonal antibody which binds to the extracellular region of the neutral amino acid transporter of the present invention may bind to any region of the extracellular region. Specifically, the monoclonal antibody which binds to the extracellular region of ASCT2 may bind to any region of the extracellular region of ASCT2. Preferably, it may bind to at least one selected from EL1 to EL5 regions in the extracellular region of ASCT2, and more preferably, it may bind to at least EL2 region in the extracellular region of ASCT2.

In the present invention, the method of evaluating the inhibitory activity of intracellular uptake of amino acids may include a method in which an antibody or an antibody fragment thereof is allowed to react with a cell, and then the inhibition of uptake of amino acids such as radioactive material-labeled alanine is evaluated using an equipment such as scintillation counter [J. Biol. Chem., 271, 14883 (1996)], or the like.

In the present invention, the method of evaluating the inhibitory activity of cell proliferation by inhibition of intracellular uptake of amino acids may include a method in which an antibody or an antibody fragment thereof is allowed to react with a cell, and then the inhibition of neutral amino acid-dependent proliferation is evaluated using a viable cell counting reagent [J. Surgical Research, 90, 149 (2000)], or the like.

In the present invention, the cell may be any cell, as long as a neutral amino acid or a neutral amino acid transporter is involved in survival or proliferation of the cell. Examples thereof may include a cell which is naturally present in the human body, a cell line established from the cell which is naturally present in the human body, a cell obtained by a gene recombination technique, or the like. The cell of the present invention is preferably a cell expressing the neutral amino acid transporter, and more preferably, a cell expressing ASCT2.

Specifically, the cell which is naturally present in the human body may be exemplified by cancer cells. Examples of the cancer may include blood cancer, breast cancer, uterine cancer, colorectal cancer, esophageal cancer, stomach cancer, ovarian cancer, lung cancer, renal cancer, rectal cancer, thyroid cancer, uterine cervix cancer, small intestinal cancer, prostate cancer, pancreatic cancer or the like. Preferably, it may include blood cancer, esophageal cancer, stomach cancer, colorectal cancer, liver cancer or prostate cancer. Examples of the blood cancer may include myeloid leukemia, lymphoid leukemia, multiple myeloma, Hodgkin's lymphoma, non-Hodgkin's lymphoma or the like.

The antibody used in the present invention may be any one of monoclonal antibody and polyclonal antibody, and preferably, a monoclonal antibody which binds to a single epitope.

The monoclonal antibody may be any monoclonal antibody of a monoclonal antibody produced from hybridomas and a genetically recombinant antibody produced by a gene recombination technique. However, in order to reduce immunogenicity in humans, a human chimeric antibody (hereinafter, simply referred to as chimeric antibody), a humanized antibody [also called human complementarity determining region (CDR)-grafted antibody] and a human antibody are preferably used.

Specifically, the monoclonal antibody of the present invention may be an antibody selected from the following (A) to (C):

(A) a monoclonal antibody in which CDR1, CDR2 and CDR3 of a heavy chain variable region (hereinafter, referred to as VH) of the antibody include the amino acid sequences shown in SEQ ID NOs:26, 27 and 28, respectively, and CDR1, CDR2 and CDR3 of a light chain variable region (hereinafter referred to as VL) of the antibody include the amino acid sequences shown in SEQ ID NOs:29, 30 and 31, respectively;

(B) a monoclonal antibody in which CDR1, CDR2 and CDR3 of VH of the antibody include the amino acid sequences shown in SEQ ID NOs:32, 33 and 34, respectively, and CDR1, CDR2 and CDR3 of VL of the antibody include the amino acid sequences shown in SEQ ID NOs:35, 36 and 37, respectively;

(C) a monoclonal antibody in which CDR1, CDR2 and CDR3 of VH of the antibody include the amino acid sequences shown in SEQ ID NOs:49, 50 and 51, respectively, and CDR1, CDR2 and CDR3 of VL of the antibody include the amino acid sequences shown in SEQ ID NOs:52, 53 and 54, respectively.

In the present invention, one embodiment of the monoclonal antibody in which CDR1, CDR2 and CDR3 of VH of the antibody include the amino acid sequences shown in SEQ ID NOs:26, 27 and 28, respectively, and CDR1, CDR2 and CDR3 of VL of the antibody include the amino acid sequences shown in SEQ ID NOs:29, 30 and 31, respectively may include an anti-ASCT2 mouse monoclonal antibody KM4008, an anti-ASCT2 human chimeric antibody cKM4008, anti-ASCT2 humanized antibodies KM4008HV2LV3 and KM4008HV10LV3 (WO 2010/008075, WO2011/087091) or the like.

In the present invention, one embodiment of the monoclonal antibody in which CDR1, CDR2 and CDR3 of VH of the antibody include the amino acid sequences shown in SEQ ID NOs:32, 33 and 34, respectively, and CDR1, CDR2 and CDR3 of VL of the antibody include the amino acid sequences shown in SEQ ID NOs:35, 36 and 37, respectively may include an anti-ASCT2 mouse monoclonal antibody KM4012, an anti-ASCT2 human chimeric antibody cKM4012 (WO 2010/008075, WO 2011/087091) or the like.

In the present invention, one embodiment of the monoclonal antibody in which CDR1, CDR2 and CDR3 of VH of the antibody include the amino acid sequences shown in SEQ ID NOs:49, 50 and 51, respectively, and CDR1, CDR2 and CDR3 of VL of the antibody include the amino acid sequences shown in SEQ ID NOs:52, 53 and 54, respectively may include an anti-ASCT2 rat monoclonal antibody KM4018, an anti-ASCT2 human chimeric antibody cKM4018 (WO 2010/008075, WO 2011/087091) or the like.

In the present invention, the monoclonal antibody refers to an antibody that is secreted by an antibody-producing cell of a single clone, and recognizes only one epitope (antigenic determinant), and the amino acid sequence (primary structure) constituting the monoclonal antibody is uniform.

The epitope refers to a single amino acid sequence, a three-dimensional structure consisting of the amino acid sequence, an amino acid sequence bound with a modification residue such as a sugar chain, glycolipid, polysaccharide lipid, an amino group, a carboxyl group, phosphate, sulfate or the like, and a three-dimensional structure consisting of said modification residue-bound amino acid sequence, which the monoclonal antibody recognizes for binding. The three-dimensional structure is a three-dimensional structure of naturally occurring proteins, which refers to a three-dimensional structure constituted with proteins that are expressed intracellularly or on a cell membrane.

In the present invention, the antibody molecule is also called immunoglobulin (hereinafter, referred to as “Ig”). Human antibodies are classified into isotypes of IgA1, IgA2, IgD, IgE, IgG1, IgG2, IgG3, IgG4 and IgM according to the difference between molecular structures. IgG1, IgG2, IgG3, and IgG4 which are relatively highly homologous to each other in terms of the amino acid sequence are also collectively called IgG.

In the present invention, the antibody molecule consists of polypeptides called a heavy chain (hereinafter, referred to as H-chain) and a light chain (hereinafter, referred to as L-chain).

Further, the H chain consists of VH and an H-chain constant region (also referred to as CH) from the N-terminal, and the L chain consists of VL and an L-chain constant region (also referred to as CL) from the N-terminal, respectively.

Furthermore, CH consists of the respective domains of a CH1 domain, a hinge domain, a CH2 domain, and a CH3 domain from the N-terminal. The CH2 domain and the CH3 domain are collectively called an Fc region or simply Fc.

The CH1 domain, hinge domain, CH2 domain, CH3 domain, and Fc region in the present invention can be identified by the number of amino acid residues from the N-terminal according to the EU index [Kabat et al., Sequences of Proteins of Immunological Interest, US Dept. Health and Human Services (1991)].

Specifically, CH1 is identified by the amino acid sequence from positions 118 to 215 in the EU index, the hinge is identified by the amino acid sequence from positions 216 to 230 in the EU index, CH2 is identified by the amino acid sequence from positions 231 to 340 of the EU index, and CH3 is identified by the amino acid sequence from positions 341 to 447 in the EU index, respectively.

A chimeric antibody refers to an antibody consists of VH and VL of an antibody derived from a non-human animal and CH and CL of a human antibody. The species of animal from which the variable region is derived is not particularly limited, as long as it is an animal that can be used for the preparation of hybridomas, such as mouse, rat, hamster, rabbit or the like.

A human chimeric antibody can be produced in the following manner: cDNAs encoding VH and VL of a non-human animal antibody are obtained, and inserted to each of expression vectors having DNAs encoding CH and CL of a human antibody so as to construct a human chimeric antibody expression vector, and these vectors are introduced to animal cells for expression. The CH of the human chimeric antibody is not particularly limited, as long as it belongs to human immunoglobulin (hereinafter, referred to as “hIg”), and those belonging to the hIgG class are preferred. The CL of the human chimeric antibody may be any one of κ class and λ class.

A humanized antibody is an antibody (human CDR-grafted antibody) which is obtained by grafting CDRs of VH and VL derived from a non-human animal antibody into appropriate positions of VH and VL of a human antibody. The human CDR-grafted antibody can be produced in the following manner: cDNA encoding the V region in which CDRs of VH and VL of a non-human animal antibody which specifically binds to the neutral amino acid transporter, in particular, ASCT2 are grafted into the frameworks (hereinafter, referred to as FR) of VH and VL of any human antibody is constructed, the cDNA is inserted to each of expression vectors having DNAs encoding CH and CL of a human antibody so as to construct a humanized antibody expression vector, and these vectors are introduced to animal cells for expression. The amino acid sequences of FRs of VH and VL of human antibody are not particularly limited, as long as they are amino acid sequences derived from human antibodies.

The CH of the humanized antibody is not particularly limited, as long as it belongs to hIg, and those belonging to the hIgG class are preferred. The CL of the humanized antibody may be any one of κ class and λ class.

The type of the antibody fragment of the present invention is not particularly limited, and examples thereof may include Fab, Fab′, F(ab′)₂, scFv, diabody, dsFv, a peptide containing CDR or the like.

Fab is an antibody fragment having a molecular weight of about 50,000 and an antigen binding activity among the fragments which are obtained by treating IgG with papain (protease). Fab can be produced by treating the antibody with papain or inserting DNA encoding Fab into an expression vector, and introducing the vector into a prokaryote or eukaryote for expression.

F(ab′)₂ is an antibody fragment having a molecular weight of about 100,000 and an antigen binding activity among the fragments which are obtained by treating IgG with pepsin (protease). F(ab′)₂ can be produced by treating the antibody with pepsin or binding Fab′ (described below) via a thioester bond or a disulfide bond.

F(ab′) is an antibody fragment having a molecular weight of about 50,000 and antigen binding activity, which is obtained by cleaving a disulfide bond at the hinge region of the above F(ab′)₂. Fab′ can be produced by treating F(ab′)₂ of the antibody with dithiothreitol or by inserting DNA encoding Fab′ of the antibody into an expression vector, and introducing the vector into a prokaryote or eukaryote for expression.

scFv is an antibody fragment having antigen binding activity, which is prepared by linking one VH and one VL using an appropriate peptide linker. scFv can be produced by obtaining cDNAs encoding VH and VL of the antibody, constructing DNA encoding scFv, inserting the DNA into an expression vector, and then introducing the expression vector into a prokaryote or eukaryote for expression.

Diabody is an antibody fragment in which scFv is dimerized. Diabody which is an antibody fragment having divalent antigen binding activity can be produced by obtaining cDNAs encoding VH and VL of the antibody, constructing DNA encoding diabody, inserting the DNA into an expression vector, and then introducing the expression vector into a prokaryote or eukaryote for expression.

dsFv is an antibody fragment obtained by binding polypeptides, in which one amino acid residue of each of VH and VL is substituted with a cysteine residue, via a disulfide bond between the cysteine residues. dsFv can be produced by obtaining cDNAs encoding VH and VL of the antibody, constructing DNA encoding dsFv, inserting the DNA into an expression vector, and then introducing the expression vector into a prokaryote or eukaryote for expression.

The peptide containing CDR is a peptide containing one or more regions of CDRs of VH or VL. The peptide containing CDR of the antibody can be produced by constructing DNA encoding CDR of VH or VL of the antibody, inserting the DNA into an expression vector, and then introducing the expression vector into a prokaryote or eukaryote for expression. The peptide containing CDR can also be produced by a chemical synthesis method such as Fmoc (fluorenylmethyloxycarbonyl) method, tBoc (t-butyloxycarbonyl) method or the like.

In the present invention, oxidative stress means a state in which the active oxygen production system becomes dominant over the active oxygen elimination system as a result of an imbalance between the production system and the elimination system, which are usually maintained in balance in cells, under various causes such as drugs, radiation, ischemia or the like. Enhancement of intracellular oxidative stress causes cell cytotoxicity, mutation by DNA base modification, DNA damage, apoptosis induction or the like.

In the present invention, the enhancement of oxidative stress may be caused by acceleration of the intracellular active oxygen production system or by inhibition of the active oxygen elimination system.

In the present invention, acceleration of the intracellular active oxygen production system means that the amounts of the amino acids or amino acid metabolites involved in the intracellular active oxygen production system are lower than those of a negative control, and inhibition of the intracellular active oxygen elimination system means that the amounts of the amino acids or amino acid metabolites involved in the intracellular active oxygen elimination system are lower than those of a negative control.

The lower amounts of the amino acids or amino acid metabolites involved in the intracellular active oxygen production system than those of the negative control, or the lower amounts of the amino acids or amino acid metabolites involved in the intracellular active oxygen elimination system than those of the negative control can be confirmed, for example, by examining whether with respect to the amino acids or amino acid metabolites involved in the active oxygen production system or the amino acids or amino acid metabolites involved in the active oxygen elimination system, a relative area of the test cell is smaller than that of the negative control in the metabolome analysis by CE-TOFMS.

In the present invention, TCA cycle is one of the process by which cells obtain energy from food. The acetyl group of acetyl CoA generated by degradation of carbohydrates, lipids, and proteins is oxidized, and the electron transport chain in the inner mitochondrial membrane is coupled, and finally, water, carbon dioxide, and adenosine triphosphate (also called ATP) are produced.

### EXAMPLE

Hereinafter, the present invention will be described in detail with reference to Examples, but the present invention is not limited to these Examples.

### Example 1

### Cell Obtainment and Culture Conditions

Gastric cancer cell line SNU-16 was obtained from the American Type Culture Collection (ATCC), and passaged using an RPMI1640 medium (manufactured by Invitrogen, Catalog No. 11875-119) supplemented with 10% Fetal Bovine Serum (manufacture by PAA, Catalog No. A15-501) twice a week at a density of 1×10⁵ cells/mL, and cultured in an incubator (manufactured by Form a, Catalog No. 3110) at 37° C. under the conditions of 5% CO₂, and then used in subsequent experiments.

In the assays, unless otherwise specified, a Dulbecco's Modified Eagle's Medium (manufactured by Sigma, Catalog No. D5030) supplemented with 3.7 g/L of Sodium bicarbonate solution (manufactured by Sigma, Catalog No. S8761), 1.0 g/L of 45% D-(+)-glucose solution in H₂O (manufactured by Sigma, Catalog No. G8769), 1 mM of sodium pyruvate solution (manufactured by Sigma, Catalog No. S8636), 0.2 mM of L-glutamine (manufactured by Nacalai Tesque, Catalog No. 1694804), and 10% dialyzed FBS (manufactured by Invitrogen, Catalog No. 26400044) (hereinafter, referred to as assay medium) was used.

### Example 2

### Actions of KM4008HV2LV3 on Intracellular Metabolic System of Cultured Cancer Cell Line

Anti-ASCT2 humanized antibody KM4008HV2LV3 binds to the extracellular region of ASCT2, and exhibits inhibitory activities on the intracellular uptake of glutamine and cell proliferation (WO 2010/008075, WO 2011/087091). In order to demonstrate molecular mechanisms of the above described activities, metabolome analysis of KM4008HV2LV3-treated and solvent-treated cells were performed by capillary electrophoresis time-of-flight mass spectrometry (CE-TOFMS).

2-1) Preparation of Cultured Cell Sample

SNU-16 cancer cell line was suspended in the assay medium at a density of 2×10⁴ cells/mL, and 30 mL thereof was seeded in a T175 flask (manufactured by Falcon, 353028). The cells were cultured for 24 hours, and then 600 μL of a medium containing 500 μg/mL of KM4008HV2LV3 (a solvent solution [10 mM sodium citrate, 262 mM D-sorbitol, 0.05 mg/mL Polysorbate 80 (pH5.7)] containing 300 μg of KM4008HV2LV3 was diluted with the assay medium to 600 μL) was added to the flask, followed by incubation at 37° C.

Further, as a negative control, 600 μL of a medium that was prepared by diluting the solvent solution of antibody using an equal amount of the assay medium was added to the flask and incubated at 37° C. to prepare the solvent-treated cells. At 2 hrs and 8 hrs after addition of the antibody and the solvent solution of the antibody, cells were detached and centrifuged at 1300 rpm for 3 minutes followed by discarding the supernatant. The precipitated fraction was suspended in 10 mL of a mannitol aqueous solution [ultrapure water added with mannitol to a final concentration of 5%].

A portion of the cell suspension was taken and the number of cells was counted, followed by centrifugation of the cell suspension. After discarding the supernatant, 1 mL of a methanol solution for metabolite extraction [a 1000-fold dilution of Internal Standard Solution 1 (10 mM, manufactured by Human Metabolome Technologies) diluted with methanol (manufactured by Wako Pure Chemical, for LC/MS, 134-14523)] was added and provided for the metabolome analysis by CE-TOFMS.

2-2) Metabolome Analysis by CE-TOFMS

The metabolome analysis by CE-TOFMS, including data analysis, was carried out by Human Metabolome Technologies, Inc. (hereinafter, also described as HMT). A method for the data analysis is described as follows. Information (mass electric charge ratio (m/z), migration time (MT), and peak area) of each peak detected by CE-TOFMS was acquired by MasterHands automatic integration software ver. 2.9.0.9 (developed at Keio University). Based on the acquired m/z and MT, the amino acid or the amino acid metabolite corresponding to each peak detected by CE-TOFMS was identified from the metabolome database of HMT Inc.

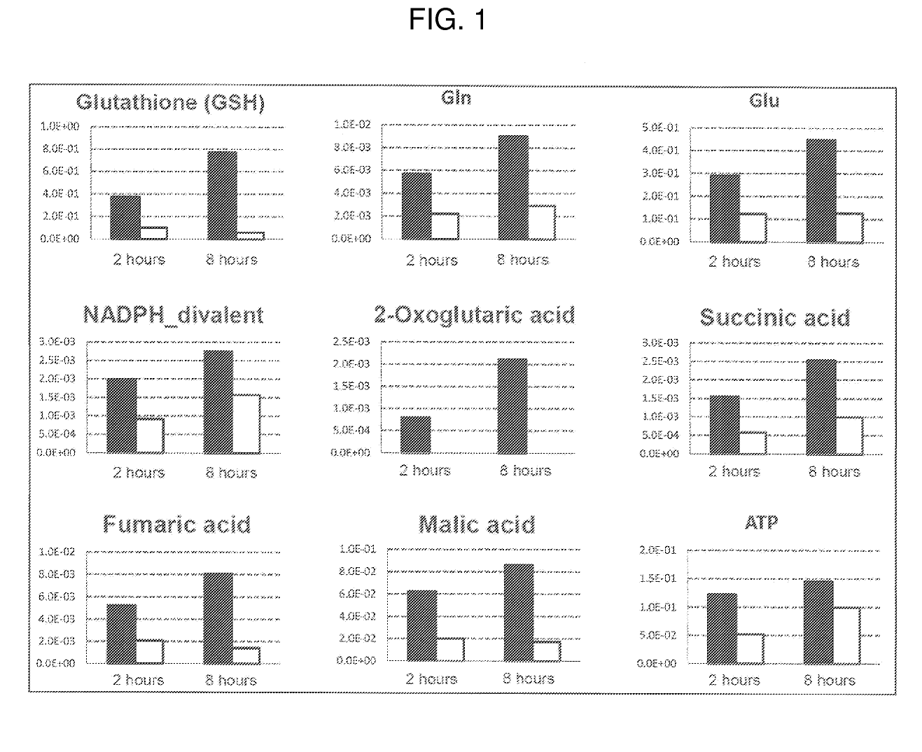

With respect to each amino acid or amino acid metabolite thus identified, a relative area shown in (peak area)/(area of internal standard material×the number of cells used in measurement) was calculated. The relative areas of the amino acids or the amino acid metabolites at 2 hrs and 8 hrs after addition of KM4008HV2LV3 or the solvent were shown in Table 1, and a graph thereof was shown in FIG. 1.

TABLE 1
Relative areas of amino acids or amino acid metabolites obtained from
metabolome analysis by CE-TOFMS at 2 hrs and 8 hrs after KM4008HV2LV3
or solvent treatment

| | Relative area | | | |
|---|---|---|---|---|
| | 2 hrs | | 8 hrs | |
| Compound name | Solvent | KM4008HV2LV3 | Solvent | KM4008HV2LV3 |
| Glutathione (GSH) | 3.7E−01 | 1.0E−01 | 7.7E−01 | 6.1E−02 |
| Gln | 5.7E−03 | 2.2E−03 | 9.0E−03 | 2.9E−03 |
| Glu | 2.9E−01 | 1.2E−01 | 4.5E−01 | 1.3E−01 |
| NADPH_divalent | 2.0E−03 | 9.1E−04 | 2.7E−03 | 1.6E−03 |
| 2-Oxoglutaric acid | 8.0E−04 | N.D. | 2.1E−03 | N.D. |
| Succinic acid | 1.6E−03 | 5.8E−04 | 2.5E−03 | 9.9E−04 |
| Fumaric acid | 5.3E−03 | 2.1E−03 | 8.1E−03 | 1.4E−03 |
| Malic acid | 6.2E−02 | 2.0E−02 | 8.6E−02 | 1.7E−02 |
| ATP | 1.2E−01 | 5.2E−02 | 1.5E−01 | 1.0E−01 |

The relative area thus obtained reflects the intracellular concentration of each amino acid or amino acid metabolite. Therefore, the relative areas of antibody treatment and solvent treatment were compared at each reaction time of 2 hrs or 8 hrs after antibody or solvent treatment, thereby evaluating changes in the intracellular concentrations of the amino acids or amino acid metabolites by the antibody treatment at each reaction time.

In the cells treated with KM4008HV2LV3, all relative areas of glutamine (Gln), glutamic acid (Glu), reduced glutathione (GSH), and nicotinamide adenine dinucleotide phosphate (NADPH) were reduced to ½ or lower of those of the solvent-treated group at 2 hrs after antibody treatment. All relative areas of the above amino acids or amino acid metabolites were also reduced to ⅔ or lower of those of the solvent-treated group at 8 hrs after antibody treatment (FIG. 1, Table 1).

As described above, the relative area of each amino acid or amino acid metabolite reflects the intracellular concentration of the amino acid or amino acid metabolite. Therefore, the results indicate that all intracellular concentrations of glutamine, glutamic acid, reduced glutathione, and NADPH are reduced by treatment of cells with KM4008HV2LV3, compared to those of the solvent-treated group, at any reaction time of 2 hrs or 8 hrs, that is, biosynthesis of these amino acids or amino acid metabolites is inhibited by antibody treatment.

In cells, glutamine is converted into glutamic acid by glutaminase, and reduced glutathione is synthesized from glutamic acid by glutathione synthetase via 5-L-glutamyl cysteine. NADPH serves as a hydrogen donor for the reduction of oxidized glutathione.

Taken together, intracellular glutamic acid is reduced by inhibition of intracellular uptake of glutamine in the KM4008HV2LV3-treated cancer cells, resulting in inhibition of glutathione synthesis. It was also found that because glutathione is an antioxidant, KM4008HV2LV3 inhibits Redox homeostasis of cancer cells (active oxygen elimination system), that is, promotes oxidative stress.

In the KM4008HV2LV3-treated cells, all the relative areas of 2-oxoglutaric acid, succinic acid, fumaric acid, malic acid, and adenosine triphosphate (ATP) were reduced to ½ or lower, and ⅘ or lower of those of the solvent-treated group at 2 hrs and 8 hrs after antibody treatment, respectively (FIG. 1, Table 1).

The above described metabolites constitute the TCA cycle which is a part of the pathways involved in the production of ATP as the cellular energy, and oxidative deamination of intracellular glutamic acid by glutamic acid dehydrogenase generates 2-oxoglutaric acid, which is used in the TCA cycle.

Taken together, in the KM4008HV2LV3-treated cancer cells, glutamic acid is reduced by inhibition of intracellular uptake of glutamine so as to reduce the TCA cycle, resulting in inhibition of the production of ATP as the cellular energy.

### Example 3

### Antibody Array Analysis Upon Addition of KM4008HV2LV3

To more clarify the molecular mechanism of the inhibitory activity of KM4008HV2LV3 on cell proliferation, in vivo signal changes by addition of KM4008HV2LV3 to cancer cells were measured using an antibody array on which a plurality of antibodies against phosphorylation proteins involved in various signal transduction pathways in vivo were spotted.

SNU-16 was seeded in 20 mL of the assay medium at a density of 1×10⁵ cells/mL, and cultured at 37° C. under the conditions of 5% CO₂ for 24 hours. Then, a medium containing KM4008HV2LV3 (a solvent solution [10 mM sodium L-glutamate, 262 mM D-sorbitol, 0.05 mg/mL PS80 (pH 5.7)] containing KM4008HV2LV3 was diluted with the assay medium) was added to a final concentration of 10 μg/mL, or an equal amount of the solvent solution of antibody was added thereto, and left at 37° C. under the conditions of 5% CO₂ for 2 hours or 24 hours.

Cell detachment and lysis, protein extraction, and biotinylation were performed using an antibody array assay kit (manufactured by Full Moon Biosystems, Catalog No. KAS02) in accordance with the manufacturer's protocols. The cell extract containing 70 μg of biotinylated protein was hybridized with the Phospho Explorer Antibody Array (manufactured by Full Moon Biosystems, Catalog No. PEX100) in accordance with the manufacturer's protocols. The biotinylated proteins on the array were reacted with Cy3 Streptavidin (manufactured by GE Healthcare, Catalog No. PA43001), and scanning was performed using a microarray scanner (manufactured by Agilent, Catalog No. G2565CA), and quantification of the obtained signals was performed.

Two spots each of 1318 types of antibodies were applied on the array, and when signals above the detection limit detected in both spots were counted, 390 antibodies and 419 antibodies were detected in the cells at 2 hours and 24 hours after KM4008HV2LV3 treatment, respectively.

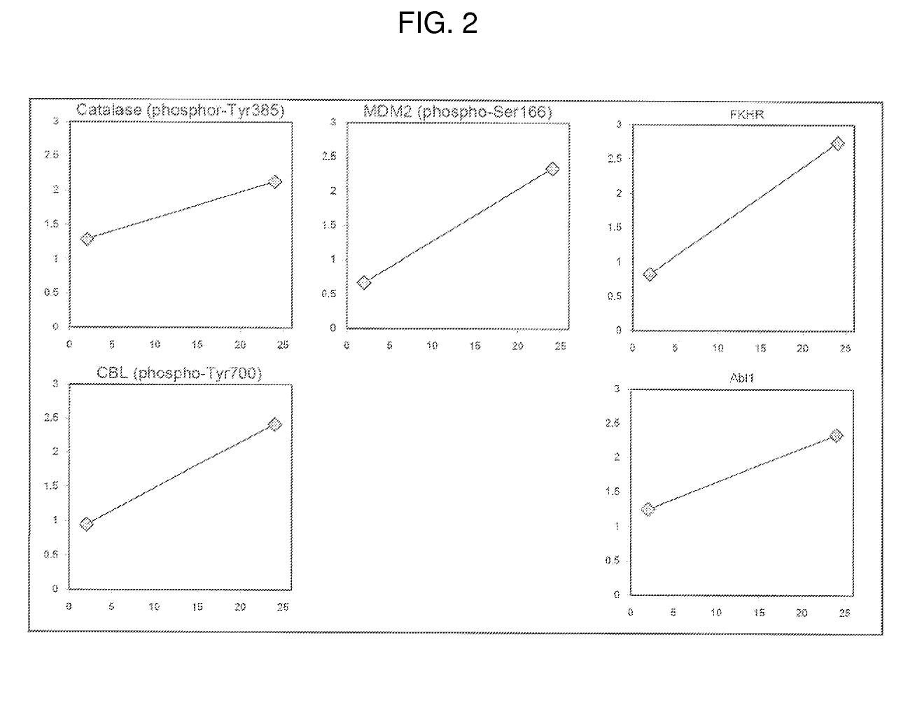

Among them, the antibodies which exhibited 2-fold or more of changes in the signal intensity between solvent-treated and KM4008HV2LV3-treated cells in both spots were 4 antibodies and 37 antibodies at 2 hours and 24 hours, respectively. Among 37 antibodies which exhibited 2-fold or more of changes in the signal intensity between solvent-treated and KM4008HV2LV3-treated cells at 24 hours after KM4008HV2LV3 treatment in both spots, changes in the signal intensity of catalase Phospho-Tyr385, Casitas B-lineage Lymphoma (CBL) Phospho-Tyr700, murine double minute 2 (MDM2) Phospho-Ser166 which are involved in oxidative stress are shown in FIG. 2.

The above signals are reported to increase upon oxidative stress [J. Biol. Chem. 278: 29667-29675, 2003, J. Biol. Chem. 282:2288-2296, 2007, Alexa Fluor (registered trade name) 647 Mouse anti-c-Cbl (pY700) technical data sheet], and the result of FIG. 2 showed that oxidative stress occurred in the KM4008HV2LV3-treated cells.

Further, increased expression levels of forkhead in rhabdomyosarcoma (FKHR) and V-abl Abelson murine leukemia viral oncogene homolog 1(Abl1) were also observed (FIG. 2). There have been no reports that these expression levels increase in response to oxidative stress. However, since FKHR is a protein that serves to induce transcription of the enzyme group eliminating active oxygen, such as catalase or the like and Abl1 is a protein that activates catalase by phosphorylation of Tyr385 (Antioxid Redox Signal. 14(4):593-605, 2011, J. Biol. Chem. 278:29667-29675, 2003), the increase of these proteins indicates promotion of oxidative stress by KM4008HV2LV3 and activation of the response pathway thereof.

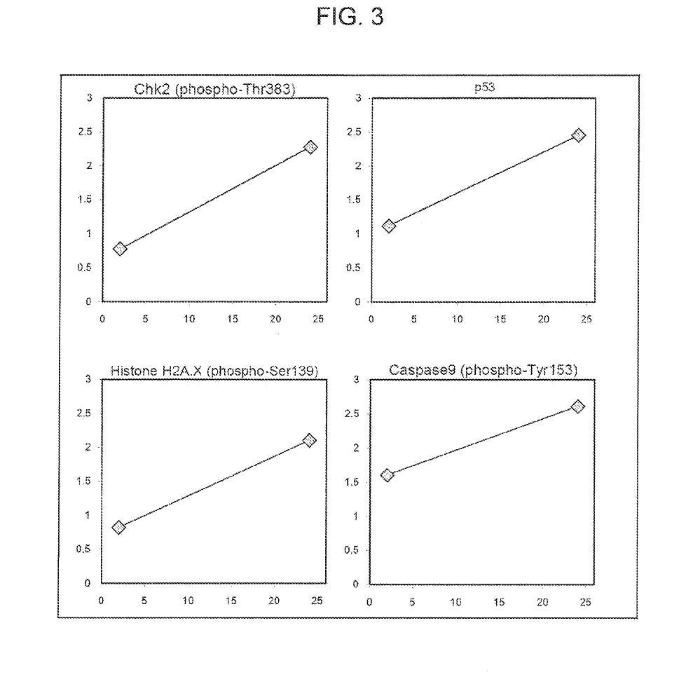

Moreover, signals of checkpoint kinase 2 (chk2) Phospho-Thr383, p53, H2A.X Phospho-Ser139, and caspase9 Phospho-Tyr153 were increased by 2-fold or more in the KM4008HV2LV3-treated cells, compared to the solvent-treated cells (FIG. 3). These proteins are known as signals that increase in response to DNA double strand break (DNA repair 8:1047-1054, 2009, Leukemia 24:679-686, 2010, J. Biol. Chem. 280:11147-11151, 2005), and therefore, this indicates that DNA double strand break occurred in the KM4008HV2LV3-treated cells.

### Example 4

### Western Blot Analysis of DNA Damage by KM4008HV2LV3 Addition

The signal increase of H2A.X Phospho-Ser139 in the KM4008HV2LV3-treated cells suggesting DNA double strand break, as shown in Example 3, was detected by Western blot. Anti-phospho-Histone H2A.X Ser139, clone JBW301 (Millipore Catalog No. 05-636) as a primary antibody and anti-mouse IgG, HRP-linked antibody (CST Catalog No. 7076) as a secondary antibody were used.

SNU-16 was seeded in 10 mL of the assay medium at a density of 2×10⁴ cells/mL, and cultured at 37° C. under the conditions of 5% CO₂ for 24 hours. Then, KM4008HV2LV3 (final concentration of 10 μg/mL), the solvent solution of antibody, or hydrogen peroxide (H₂O₂) (final concentration of 50 μM) was added. The cells were left at 37° C. under the conditions of 5% CO₂ for 8 hours, and the suspending cells were recovered together with the culture supernatant.

The adherent cells were washed with D-PBS(−) (manufactured by Nacalai Tesque, Catalog No. 14249-95), and then 1.5 mL of TrypLE select (manufactured by Invitrogen, Catalog No. 12563-011) was added and left at 37° C. under the conditions of 5% CO₂ for 5 minutes for detachment, and then mixed with the suspending cells recovered in advance to be used in the subsequent manipulations.

The recovered cells were washed with D-PBS and then centrifuged, and suspended in a solution that is prepared by adding 1/100 of protease inhibitor cocktail (manufactured by Merck, Catalog No. 535142) and phosphatase inhibitor cocktail (manufactured by Nacalai Tesque, Catalog No. 07574-61) to an RIPA buffer (manufactured by Takara Bio, Catalog No. P75682) for lysis.

The cell lysate thus obtained was left on ice for 15 minutes, followed by centrifugation at 15,000 rpm for 15 minutes. The protein concentration in the supernatant thus obtained was determined using a BCA protein assay kit (manufactured by Pierce, Catalog No. 23225). The cell lysate was prepared at a concentration of 1.2 μg/μL using the RIPA buffer, and then ⅕ of the sample buffer (for SDS-PAGE, 6-fold concentration, containing a reducing agent) (manufactured by Nacalai Tesque, Catalog No. 09499-14) was added and boiled at 100° C. for 5 minutes, and used as a cell extract. The cell extract containing 10 μg of protein was electrophoresed in a 5-20% gradient gel (manufactured by ATTO, ePAGEL, Catalog No. 2331730) to separate proteins. The proteins were transferred onto the PVDF membrane (manufactured by Millipore, Catalog No. IPVH304F0) using EzBlot (manufactured by ATTO, Catalog No. AE-1460), and then blocking was performed by block ACE (manufactured by DS Pharma Biomedical, Catalog No. UK-B40).

After terminating the blocking, the membrane was reacted with the primary antibody that is prepared by using Can Get Signal (manufactured by TOYOBO, Catalog No. NKB-101), and washed with 0.05% Tween-20-added Tris Buffered Serine (TBS-T), and then reacted with the secondary antibody that is prepared by using Can Get Signal. After washing with TBS-T, the membrane was put in Supersignal West Dura (manufactured by Thermo Fisher, Catalog No. 34075), and chemical luminance was detected using a Lumino image analyzer (manufactured by Fuji Film, LAS-3000).

As a result, increased phosphorylation signals of H2A.X Ser139 were observed in the KM4008HV2LV3-treated and H₂O₂-treated cells, compared to the solvent-treated cells. As in Example 3, it was found that DNA double strand break occurred in the KM4008HV2LV3-treated cells.

### Example 5

### Cell Cycle Change by KM4008HV2LV3

The possibility of cell cycle arrest in the G1 phase or cell death was considered as the cause of cell proliferation inhibition by KM4008HV2LV3. Therefore, cells were stained with propidium iodide (PI), and fluorescence intensity was measured using a flow cytometer to evaluate cell cycle change by KM4008HV2LV3 treatment.

SNU-16 was seeded in 10 mL of the assay medium at a density of 2×10⁴ cells/mL, and cultured at 37° C. under the conditions of 5% CO₂. After culture for 24 hours, a medium containing KM4008HV2LV3 (a solvent solution (10 mM Sodium L-glutamate, 262 mM D-Sorbitol, 0.05 mg/mL PS80 pH 5.7) containing KM4008HV2LV3 was diluted with the assay medium) (final concentration of 10 μg/mL), an equal amount of the solvent solution of antibody, or hydrogen peroxide (manufactured by WAKO, Catalog No. 081-04215) (final concentration of 50 μM) was added.

After culture for 24 hours, the suspending cells were recovered together with the culture supernatant. The adherent cells were washed with D-PBS(−) (manufactured by Nacalai Tesque, Catalog No. 14249-95), and then 1.5 mL of TrypLE select (manufactured by Invitrogen, Catalog No. 12563-011) was added and left at 37° C. under the conditions of 5% CO₂ for 5 minutes, and then recovered and mixed with the suspending cells recovered in advance to be used in the subsequent manipulations.

After centrifugation, the cells were washed with D-PBS(−), and the pellet was suspended in 5% D-(+)-glucose. 100% ethanol (manufactured by Nacalai Tesque, Catalog No. 14713-95) was added thereto to a final concentration of 70%, and fixed at −30° C.

After fixation, the cells were washed with D-PBS(−) twice and the pellet was suspended in a PI/RNase staining buffer (manufactured by BD Bioscience, Catalog No. 550825), and left for 30 minutes in the dark. Then, fluorescence intensity was measured using a flow cytometer (manufactured by BD bioscience, FACS Calibur).

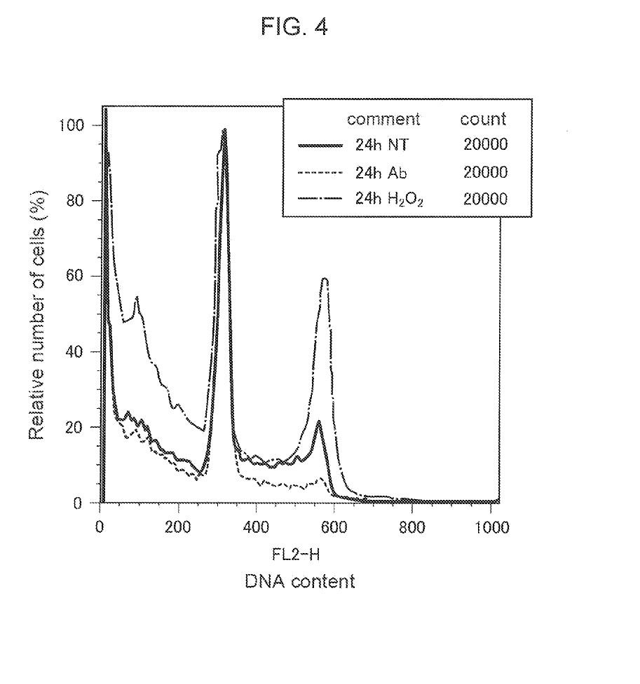

The acquired data were analyzed by FlowJo (manufactured by Tree Star Inc.) and the results are shown in FIG. 4. In FIG. 4, fluorescence intensity of the horizontal axis represents the DNA amount contained in the cells, the peak around the fluorescence intensity 600 represents cell populations of the G2/M phase, and the peak around the fluorescence intensity 300 represents cell populations of the G1 phase. In addition, cell populations showing the lower fluorescence intensity than that of the G1 phase are those that undergo cell death. Cell populations that exist between the peaks representing cell populations of the G1 phase and the G2/M phase are cell population of the S phase. A ratio of the peak height reflecting the number of cells in each cell population with respect to each histogram upon addition of antibody, solvent or hydrogen peroxide was acquired.

As a result, it was found that a larger number of cells in the G1 phase and a smaller number of cells in the S and the G2/M phases were observed in KM4008HV2LV3-treated cells, compared to the solvent-treated cells, indicating cell cycle arrest in the G1 phase (FIG. 4). In addition, cells having a smaller amount of DNA than that of the G1 phase were increased in the KM4008HV2LV3-treated cells, indicating cell death (FIG. 4).

Furthermore, in order to confirm the cell cycle arrest in the G1 phase at the protein level, Western blot was performed in the same manner as in Example 4, and the amount of p21 protein was analyzed. It has been known that p21 expression is induced by p53 and p21 interacts with cyclin E and cyclin dependent kinase 2 (CDK2) complex or cyclin D and cyclin dependent kinase 4/6 (CDK4/6) complex to inhibit its function, thereby inhibiting S phase progression (Proc Soc Exp Biol Med. 213:138-149, 1996).

p21(C19)antibody (Santa Cruz Catalog No. sc-397) as a primary antibody and Anti-rabbit IgG, HRP-linked Antibody (CST Catalog No. 7074) as a secondary antibody were used. The result of Western blot showed that p21 signal was increased in the KM4008HV2LV3-treated cells, compared to the solvent-treated cells, indicating cell cycle arrest in the G1 phase, which is consistent with the results of cell cycle analysis.

### Example 6

### Analysis of Cell Death Caused by KM4008HV2LV3

Based on the result of Example 5 showing that cell death occurred in the KM4008HV2LV3-treated cells, cells were stained with Annexin V, and fluorescence intensity was measured using a flow cytometer to analyze cell death in the KM4008HV2LV3-treated cells.

SNU-16 cancer cells were seeded in 10 mL of the assay medium at a density of 2×10⁴ cells/mL, and cultured at 37° C. under the conditions of 5% CO₂. After culture for 24 hours, a medium containing KM4008HV2LV3 (a solvent solution containing KM4008HV2LV3 (10 mM Sodium L-glutamate, 262 mM D-Sorbitol, 0.05 mg/mL PS80 pH 5.7) was diluted with the assay medium) (final concentration of 10 μg/mL), the solvent solution of antibody, or hydrogen peroxide (manufactured by WAKO, Catalog No. 081-04215) (final concentration of 50 μM) was added.

After culture for 72 hours, the suspending cells were recovered together with the culture supernatant. The adherent cells were washed with D-PBS(−) (manufactured by Nacalai Tesque, Catalog No. 14249-95), and then 1.5 mL of TrypLE select (manufactured by Invitrogen, Catalog No. 12563-011) was added and left at 37° C. under the conditions of 5% CO₂ for 5 minutes, and then recovered and mixed with the suspending cells recovered in advance to be used in the subsequent manipulations.

The recovered cells were washed with D-PBS(−), followed by centrifugation. The pellet was suspended in 100 μL of Annexin V Binding Buffer (BD Catalog No. 556454). 5 μL of annexin V-FITC (manufactured by BD, Catalog No. 556420) and 0.5 μL of propidium iodide (manufactured by Invitrogen, Catalog No. P3566) were added thereto and left in the dark for 15 minutes.

400 μL of annexin V Binding Buffer was added, and then fluorescence intensity was measured using a flow cytometer (manufactured by BD bioscience, FACS Calibur). FlowJo (manufactured by Tree Star Inc.) was used for data analysis.

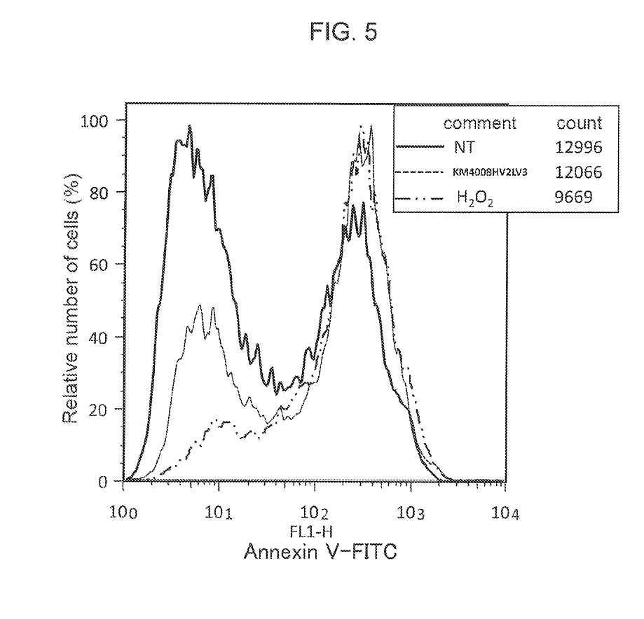

As a result, it was found that when cells were cultured in a medium containing 0.2 mM of L-glutamine for 72 hours, about half of SNU-16 cells was annexin V-positive. Furthermore, the percentage of annexin V-positive cells in the KM4008HV2LV3-treated cells was found to be higher than that of the solvent-treated cells (FIG. 5), indicating that cell death of SNU-16 was caused by KM4008HV2LV3 treatment.

### Example 7

### Recovery of KM4008HV2LV3-Caused Cell Proliferation Inhibition by Antioxidant

Based on the results of Examples 2 and 3 showing that oxidative stress occurred in the KM4008HV2LV3-treated cells, a flow cytometer was used to examine changes in the proliferation of KM4008HV2LV3-treated cells in the presence or absence of an antioxidant N-acetylcysteine (NAC) in order to demonstrate a causal relationship with cell proliferation inhibition.

SNU-16 was seeded in 1 mL of the assay medium at a density of 2×10⁴ cells/mL, and cultured at 37° C. under the conditions of 5% CO₂. After culture for 24 hours, KM4008HV2LV3 (final concentration of 10 μg/mL), the solvent solution of antibody, hydrogen peroxide (final concentration of 50 μM) or NAC (manufactured by Sigma, Catalog No. A9165) (final concentration of 5 mM) was added thereto. The cells were cultured at 37° C. under the conditions of 5% CO₂ for 48 or 72 hours, and the suspending cells were recovered together with the culture supernatant.

The adherent cells were washed with D-PBS(−) (manufactured by Nacalai Tesque, Catalog No. 14249-95), and then 1.5 mL of TrypLE select (manufactured by Invitrogen, Catalog No. 12563-011) was added and left at 37° C. under the conditions of 5% CO₂ for 5 minutes, and then recovered and mixed with the suspending cells recovered in advance to be used in the subsequent manipulations.

The recovered cells were washed with D-PBS (Nacalai Tesque, Catalog No. 14249-95), and suspended in 1 mL of 3% fetal bovine serum in D-PBS. Flow-Count (manufactured by Beckman Coulter, Catalog No. 7547053) and propidium iodide (manufactured by Invitrogen, Catalog No. P3566) were added and a flow cytometer (manufactured by BD bioscience, FACS calibur) was used to count the number of live cells.

FlowJo (manufactured by Tree Star Inc.) was used for analysis of the acquired data. Cells were gated by front scatter (FSC) and side scatter (SSC), and the number of cells was calculated from the ratio of the number of live cells to the number of beads of known concentration.

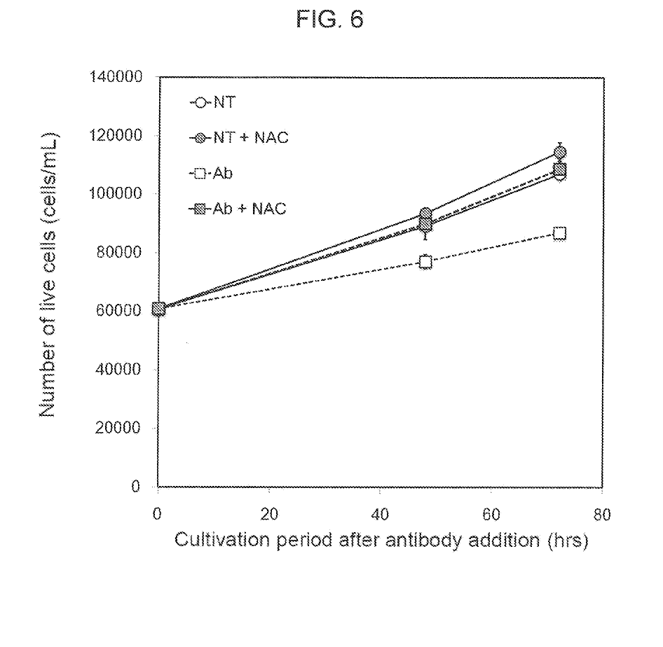

As a result, cell proliferation of the KM4008HV2LV3-treated cells was inhibited, compared to the solvent-treated cells. It was also revealed that inhibition of cell proliferation by KM4008HV2LV3 treatment was recovered by addition of NAC (FIG. 6), indicating that inhibition of cell proliferation by KM4008HV2LV3 treatment was induced by oxidative stress.

While the invention has been described in detail and with reference to specific embodiments thereof, it will be apparent to one skill in the art that various changes and modifications can be made therein without departing from the spirit and scope thereof.

This application is based on U.S. provisional application No. 61/717,703, filed on Oct. 24, 2012, the entire contents of which are incorporated hereinto by reference. All references cited herein are incorporated in their entirety.

SEQ ID NO:3—Description of artificial sequence: nucleotide sequence of ASCT2 primer (Fw Catalog No. 1)

SEQ ID NO:4—Description of artificial sequence: nucleotide sequence of ASCT2 primer (Rv Catalog No. 1)

SEQ ID NO:5—Description of artificial sequence: nucleotide sequence of ASCT2-myc/His

SEQ ID NO:6—Description of artificial sequence: amino acid sequence of ASCT2-myc/His

SEQ ID NO:7—Description of artificial sequence: nucleotide sequence of N-ASCT2 primer (Catalog No. 1)

SEQ ID NO:8—Description of artificial sequence: nucleotide sequence of N-ASCT2 primer (Catalog No. 2)

SEQ ID NO:9—Description of artificial sequence: nucleotide sequence of N-ASCT2 primer (Catalog No. 3)

SEQ ID NO:10—Description of artificial sequence: nucleotide sequence of N-ASCT2 primer (Catalog No. 4)

SEQ ID NO:11—Description of artificial sequence: nucleotide sequence of C-ASCT2-myc/His primer (Fw Catalog No. 2)

SEQ ID NO:12—Description of artificial sequence: nucleotide sequence of C-ASCT2-myc/His primer (Rv Catalog No. 2)

SEQ ID NO:13—Description of artificial sequence: nucleotide sequence of C-ASCT2-myc/His primer (Rv Catalog No. 3)

SEQ ID NO:14—Description of artificial sequence: amino acid sequence of ASCT2 peptide (2-16, Cys)

SEQ ID NO:15—Description of artificial sequence: nucleotide sequence of primer (mG3a2)

SEQ ID NO:16—Description of artificial sequence: nucleotide sequence of primer (mG2ba1)

SEQ ID NO:17—Description of artificial sequence: nucleotide sequence of primer (mKa1)

SEQ ID NO:38—Description of artificial sequence: nucleotide sequence of cKM4008VH/cKW4012VH primer (Fw)

SEQ ID NO:39—Description of artificial sequence: nucleotide sequence of cKM4008VH primer (Rv)

SEQ ID NO:40—Description of artificial sequence: nucleotide sequence of cKM4008VL/cKM4012VL primer (Fw)

SEQ ID NO:41—Description of artificial sequence: nucleotide sequence of cKM4008VL/cKM4012VL primer (Rv)

SEQ ID NO:42—Description of artificial sequence: nucleotide sequence of cKM4012VH primer (Rv)

SEQ ID NO:43—Description of artificial sequence: nucleotide sequence of primer (rG2a)

SEQ ID NO:44—Description of artificial sequence: nucleotide sequence of primer (rKa2)

SEQ ID NO:55—Description of artificial sequence: nucleotide sequence of cKM4018VH primer (Fw)

SEQ ID NO:56—Description of artificial sequence: nucleotide sequence of cKM4018VH primer (Rv)

SEQ ID NO:57—Description of artificial sequence: nucleotide sequence of cKM4018VL primer (Fw)

SEQ ID NO:58—Description of artificial sequence: nucleotide sequence of cKM4018VL primer (Rv)

SEQ ID NO:59—Description of artificial sequence: nucleotide sequence of mouse ASCT2-myc/His

SEQ ID NO:60—Description of artificial sequence: amino acid sequence of mouse ASCT2-myc/His

SEQ ID NO:61—Description of artificial sequence: nucleotide sequence of mouse ASCT2-myc/His primer (Fw)

SEQ ID NO:62—Description of artificial sequence: nucleotide sequence of mouse ASCT2-myc/His primer (Rv)

SEQ ID NO:63—Description of artificial sequence: nucleotide sequence of human ASCT2 primer (Fw) for introducing mutation of NcoI site

SEQ ID NO:64—Description of artificial sequence: nucleotide sequence of human ASCT2 primer (Rv) for introducing mutation of NcoI site

SEQ ID NO:65—Description of artificial sequence: nucleotide sequence of human ASCT2 primer (Fw) for introducing BamIII site

SEQ ID NO:66—Description of artificial sequence: nucleotide sequence of human ASCT2 primer (Rv) for introducing BamIII site

SEQ ID NO:67—Description of artificial sequence: nucleotide sequence of mouse ASCT2 primer (Fw) for introducing mutation of NcoI site

SEQ ID NO:68—Description of artificial sequence: nucleotide sequence of mouse ASCT2 primer (Rv) for introducing mutation of NcoI site

SEQ ID NO:69—Description of artificial sequence: nucleotide sequence of human ASCT2 EL4 primer (Fw) for replacing mouse type

SEQ ID NO:70—Description of artificial sequence: nucleotide sequence of human ASCT2 EL4 primer (Rv) for replacing mouse type

SEQ ID NO:71—Description of artificial sequence: amino acid sequence of KM4008 HV0

SEQ ID NO:72—Description of artificial sequence: amino acid sequence of KM4008 LV0

SEQ ID NO:73—Description of artificial sequence: nucleotide sequence of KM4008 HV0

SEQ ID NO:74—Description of artificial sequence: nucleotide sequence of KM4008 LV0

SEQ ID NO:75—Description of artificial sequence: nucleotide sequence of HV2

SEQ ID NO:76—Description of artificial sequence: amino acid sequence of HV2

SEQ ID NO:77—Description of artificial sequence: nucleotide sequence of HV4

SEQ ID NO:78—Description of artificial sequence: amino acid sequence of HV4

SEQ ID NO:79—Description of artificial sequence: nucleotide sequence of HV7

SEQ ID NO:80—Description of artificial sequence: amino acid sequence of HV7

SEQ ID NO:81—Description of artificial sequence: nucleotide sequence of HV10

SEQ ID NO:82—Description of artificial sequence: amino acid sequence of HV10

SEQ ID NO:83—Description of artificial sequence: nucleotide sequence of LV3

SEQ ID NO:84—Description of artificial sequence: amino acid sequence of LV3

SEQ ID NO:87—Description of artificial sequence: nucleotide sequence of HV2 synthetic gene

SEQ ID NO:88—Description of artificial sequence: nucleotide sequence of LV3 synthetic gene

SEQ ID NO:89—Description of artificial sequence: nucleotide sequence of HV10 synthetic gene

What is claimed is:

1. An intracellular oxidative stress promoter, comprising a monoclonal antibody having an inhibitory activity of intracellular uptake of neutral amino acids or an antibody fragment thereof as an active ingredient.

2. The intracellular oxidative stress promoter according to claim 1, wherein the neutral amino acid is glutamine.

3. The intracellular oxidative stress promoter according to claim 1, wherein the monoclonal antibody is an antibody which binds to the extracellular region of a neutral amino acid transporter.

4. The intracellular oxidative stress promoter according to claim 1, wherein the monoclonal antibody is an antibody which binds to the extracellular region of a system ASC amino acid transporter 2 (ASCT2).

5. The intracellular oxidative stress promoter according to claim 1, wherein the monoclonal antibody is an antibody selected from (A) to (C):
(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;
(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;
(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

6. The intracellular oxidative stress promoter according to claim 1, wherein the cell is a cancer cell.

7. A cell proliferation inhibitor, comprising a monoclonal antibody having an inhibitory activity of intracellular uptake of neutral amino acids or an antibody fragment thereof as an active ingredient and promoting intracellular oxidative stress.

8. The cell proliferation inhibitor according to claim 7, wherein the neutral amino acid is glutamine.

9. The cell proliferation inhibitor according to claim 7, wherein the monoclonal antibody is an antibody which binds to the extracellular region of a neutral amino acid transporter.

10. The cell proliferation inhibitor according to claim 7, wherein the monoclonal antibody is an antibody which binds to the extracellular region of ASCT2.

11. The cell proliferation inhibitor according to claim 7, wherein the monoclonal antibody is an antibody selected from (A) to (C):
(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;
(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;
(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

12. The cell proliferation inhibitor according to claim 7, wherein the cell is a cancer cell.

13. A method for promoting intracellular oxidative stress, comprising administration of a monoclonal antibody having an inhibitory activity of intracellular uptake of neutral amino acids or an antibody fragment thereof.

14. The method according to claim 13, wherein the neutral amino acid is glutamine.

15. The method according to claim 13, wherein the monoclonal antibody is an antibody which binds to the extracellular region of a neutral amino acid transporter.

16. The method according to claim 13, wherein the monoclonal antibody is an antibody which binds to the extracellular region of a system ASC amino acid transporter 2 (ASCT2).

17. The method according to claim 13, wherein the monoclonal antibody is an antibody selected from (A) to (C):
(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;
(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;
(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

18. The method according to claim 13, wherein the cell is a cancer cell.

19. A method for inhibiting cell proliferation, comprising administration of a monoclonal antibody having an inhibitory activity of intracellular uptake of neutral amino acids or an antibody fragment thereof and promotion of intracellular oxidative stress.

20. The method according to claim 19, wherein the neutral amino acid is glutamine.

21. The method according to claim 19, wherein the monoclonal antibody is an antibody which binds to the extracellular region of a neutral amino acid transporter.

22. The method according to claim 19, wherein the monoclonal antibody is an antibody which binds to the extracellular region of ASCT2.

23. The method according to claim 19, wherein the monoclonal antibody is an antibody selected from (A) to (C):
(A) a monoclonal antibody in which CDR1, CDR2 and CDR3 of VH of the antibody comprise the amino acid sequences shown in SEQ ID NOs:26, 27 and 28, respectively, and CDR1, CDR2 and CDR3 of VL of the antibody comprise the amino acid sequences shown in SEQ ID NOs:29, 30 and 31, respectively;
(B) a monoclonal antibody in which CDR1, CDR2 and CDR3 of VH of the antibody comprise the amino acid sequences shown in SEQ ID NOs:32, 33 and 34, respectively, and CDR1, CDR2 and CDR3 of VL of the antibody comprise the amino acid sequences shown in SEQ ID NOs:35, 36 and 37, respectively;
(C) a monoclonal antibody in which CDR1, CDR2 and CDR3 of VH of the antibody comprise the amino acid sequences shown in SEQ ID NOs:49, 50 and 51, respectively, and CDR1, CDR2 and CDR3 of VL of the antibody comprise the amino acid sequences shown in SEQ ID NOs:52, 53 and 54, respectively.

24. The method according to claim 19, wherein the cell is a cancer cell.
